(19) United States
(12) Patent Application Publication
(10) Pub. No.: US 2018/0080023 A1
(43) Pub. Date: Mar. 22, 2018

(54) MIRNA MIMETICS AND THEIR USE IN TREATING SENSORY CONDITIONS

(71) Applicant: MIRAGEN THERAPEUTICS, INC., Boulder, CO (US)

(72) Inventors: Aimee L. JACKSON, Boulder, CO (US); Christina M. Dalby, Boulder, CO (US)

(21) Appl. No.: 15/558,505

(22) PCT Filed: Mar. 16, 2016
(86) PCT No.: PCT/US16/22645

§ 371 Date: Sep. 14, 2017

Related U.S. Application Data
(60) Provisional application No. 62/133,590, filed on Mar. 16, 2015.

(51) Int. Cl. C12N 15/113; A61K 31/7088

(52) U.S. Cl. CPC C12N 15/113; A61K 31/7088; C12N 2310/141; C12N 2310/32; A61K 48/00; C12N 2310/3515; C12N 2310/315; C12N 2320/31; C12N 2310/321

(57) ABSTRACT

The present invention provides microRNA mimetic compounds that mimic the function or activity of miR-96, miR-182, and/or miR-183. The microRNA mimetic compounds of the invention comprise a first strand of about (22) to about (26) ribonucleotides comprising a mature miR-96, miR-182, or miR-183 sequence; and a second strand of about (20) to about (24) ribonucleotides comprising a sequence that is substantially complementary to the first strand and having at least one modified nucleotide, wherein the first strand has a 3′ nucleotide overhang relative to the second strand. The invention additionally provides expression vectors comprising a polynucleotide(s) encoding one or more of miR-96, miR-182, and miR-183. The invention also provides methods of treating ophthalmological or otic conditions by administering the microRNA mimetic compounds of miR-96, miR-182, and/or miR-183 and/or an expression vector encoding at least one of miR-96, miR-182, and miR-183 to a subject in need thereof.

### CROSS-REFERENCE TO RELATED APPLICATIONS

The present application claims the benefit of priority to U.S. Provisional Application No. 62/133,590, filed on Mar. 16, 2015, the contents of which are hereby incorporated by reference herein in their entirety.

### FIELD OF THE INVENTION

The present invention provides microRNA mimetic compounds that mimic the function or activity of miR-96, miR-182, and/or miR-183. The invention also provides expression vectors comprising a polynucleotide(s) encoding one or more of miR-96, miR-182, and miR-183. The invention further provides compositions comprising miR-96, miR-182, and/or miR-183 mimetic compounds or expression vectors encoding miR-96, miR-182, and/or miR-183, and methods of treating ophthalmological and otic conditions using the microRNA mimetic compounds or expression vectors encoding them.

### BACKGROUND

MicroRNAs are small, endogenous, noncoding RNAs that act as posttranscriptional repressors of gene expression. MicroRNAs have unique expression profiles in the developing and adult retina and are involved in normal development and functions of the retina (Ryan et al., Mol Vis 12:1175-1184, 2006; Xu et al., J Biol Chem 282(34):25053-25066, 2007). MiRNAs are dysregulated in the retina of retinal degenerative mouse models, suggesting their potential involvement in retinal degeneration (Loscher et al., Genome Biol 8(11):R248, 2007; Loscher et al., Exp Eye Res 87(6):529-534 2008). Conditional inactivation of dicer, an RNase III endonuclease required for miRNA maturation in cytosol, in the mouse retina resulted in alteration of retinal differentiation and optic-cup patterning, increased cell death, and disorganization of axons of retinal ganglion cells (Pinter & Hindges, PLoS ONE 5(4):e10021, 2010; Davis & Ashery-Padan, Development 138(1): 127-138, 2011; Georgi & Reh, J Neurosci 30(11):4048-4061, 2010), suggesting that miRNAs are important for normal development and functions of the mammalian retina. However, in vivo functions of individual miRNAs in the retina are still largely unknown.

The microRNA-183/96/182 cluster is expressed in the retina and other sensory organs. A knock-out mouse model of the miR-183/96/182 cluster showed that that the inactivation of the cluster during development results in the early-onset and progressive synaptic defects of the photoreceptors and progressive retinal degeneration (Lumayag et al., Proc Natl Acad Sci, 110(6):E507-16, 2013). On the other hand, a transgenic anti-miRNA “sponge” mouse model that reduced the activities of all three miRNAs in the miR-183/96/182 cluster showed increased bright-light induced retinal degeneration; however, no histological or functional defects of the retina were observed under normal lighting conditions (Zhu et al., J Biol Chem., 286(36):31749-60, 2011). Thus, the role of the miR-183/96/182 cluster in adult retina remains uncertain.

While many studies have shown therapeutic efficacy using single-stranded miRNA inhibitors called antimiRs, efforts to restore or increase the function of a miRNA have been lagging behind (van Rooij et al, Cir Res, 110:496-507, 2012). Currently, miRNA function can be increased either by viral overexpression or by using synthetic double-stranded miRNAs. The use of adeno-associated viruses (AAV) to drive expression of a given miRNA for restoring its activity in vivo has shown to be effective in a mouse model of hepatocellular and lung carcinoma (Kasinski & Slack, Cancer Res, 72: 5576-5587, 2012; Kota et al., Cell, 137: 1005-1017, 2009) and spinal and bulbar muscular atrophy (Miyazaki et al., Nat Med., 18(7):1136-41, 2012). The use of synthetic oligonucleotide-based approaches to increase miRNA levels has not been well explored yet. The present invention provides synthetic oligonucleotides as well as virally expressed polynucleotides that mimic the activity of miR-96, miR-182 and/or miR-183 to restore eye and ear function.

### SUMMARY OF THE INVENTION

The present invention provides microRNA mimetic compounds that mimic the function or activity of microRNAs, in particular, the function or activity of miR-96, miR-182, and/or miR-183. In some embodiments, the microRNA mimetic compounds of the invention include synthetic oligonucleotides. In other embodiments, the invention provides expression vectors comprising a polynucleotide(s) encoding one or more of miR-96, miR-182, and miR-183 for expression in a mammalian cell for treating eye or ear dysfunction.

In certain embodiments, the microRNA mimetic compounds of the invention comprise synthetic oligonucleotides comprising a first strand and a second strand, where the two strands form a double stranded region that is fully or partially complementary. In various embodiments, the first strand or the antisense strand of the microRNA mimetic compound comprises the sequence of a mature miR-96, miR-182, or miR-183 and the second strand or the sense strand comprises a sequence that is substantially complementary to the first strand and has at least one modified nucleotide.

The microRNA mimetic compounds of the invention mimic the function or activity of a mature, naturally-occurring miR-96, miR-182, or miR-183 microRNA and show enhanced resistance to the nuclease digestion of the antisense strand, improved ability to load the antisense strand into the miRNA-induced silencing complex (miRISC), and/or rapid degradation of the sense strand.

In some embodiments, the first strand of the miRNA mimetic compound is about 19, 20, 21, 22, 23, 24, 25, 26, 27, or 28 nucleotides in length and comprises the sequence of a mature miR-96, miR-182, or miR-183 and the second strand is about 18, 19, 20, 21, 22, 23, 24, 25, 26, or 27 nucleotides in length and comprises a sequence that is substantially complementary to the first strand, wherein the second strand comprises at least one modified nucleotide. In certain embodiments, the first strand is about 22 to about 26 nucleotides in length and the second strand is about 20 to about 24 nucleotides in length. In some embodiments, the first strand is about 22 to about 26 nucleotides in length and the second strand is about 20 to about 26 nucleotides in length.

In some embodiments, the sequence of the first strand of the microRNA mimetic compounds of the invention is about 80%, 85%, 90%, 91%, 92%, 93%, 94%, 95%, 96%, 97%, 98%, or 99%, inclusive of values therebetween, identical to the mature miR-96, miR-182, or miR-183 sequence. In other embodiments, the sequence of the first strand is completely (100%) identical to the mature miR-96, miR-182, or miR-183 sequence. In some embodiments, the first strand may comprise a 3′ nucleotide overhang relative to the second strand. In other embodiments, the second strand may comprise a 3′ nucleotide overhang relative to the first strand. The 3′ overhang on the first or the second strand may comprise about 1, 2, 3, or 4 nucleotides. In certain embodiments, the 3′ overhang is about 1 or 2 nucleotides in length. In some embodiments, the first and the second strand contain the same number of nucleotides, i.e., there is no overhang on either strand. In some embodiments, the overhang may be present on the 5′ end of the first or the second strand.

In one embodiment, the sequence of the second strand is fully complementary (100%) to the sequence of the first strand. In another embodiment, the sequence of the second strand is substantially complementary, such as about 75, 76, 77, 78, 79, 80, 81, 82, 83, 84, 85, 86, 87, 88, 89, 90, 91, 92, 93, 94, 95, 96, 97, 98, 99, or 100%, inclusive of values therebetween, complementary to the first strand. In still another embodiment, the sequence of the second strand may contain about 1, 2, 3, 4, or 5 mismatches relative to the first strand.

In some embodiments, the first strand and/or the second strand may comprise one or more modified nucleotides. In some embodiments, the first strand comprising a mature miR-96, miR-182, or miR-183 sequence contains one or more modified nucleotides, such as one or more 2′-fluoro modified nucleotides. In certain embodiments, the first strand contains at least one 2′-fluoro nucleotide. In some embodiments, the second strand comprising a sequence that is substantially complementary to the sequence of the first strand comprises at least one modified nucleotide, such as a 2′-fluoro or 2′-O-methyl modified nucleotide. In certain embodiments, the at least one modified nucleotide in the second strand is a 2′-O— methyl modified nucleotide.

In some embodiments, the present invention provides a microRNA mimetic compound comprising a first strand of about 22 to about 26 ribonucleotides comprising a mature miR-96, miR-182, or miR-183 sequence; and a second strand of about 20 to about 24 ribonucleotides comprising a sequence that is substantially complementary to the first strand and having at least one modified nucleotide, wherein the first strand has a 3′ nucleotide overhang relative to the second strand. In some other embodiments, the present invention provides a microRNA mimetic compound comprising a first strand of about 22 to about 26 ribonucleotides comprising a mature miR-96, miR-182, or miR-183 sequence; and a second strand of about 20 to about 26 ribonucleotides comprising a sequence that is substantially complementary to the first strand and having at least one modified nucleotide, wherein the first or the second strand has a 3′ nucleotide overhang.

The invention further provides expression vectors comprising a polynucleotide encoding miR-96, miR-182, and/or miR-183 positioned for expression in a mammalian cell for use as a therapeutic agent for the treatment of ophthalmological or otic disorders. For example, the sequence encoding miR-96, miR-182, and/or miR-183 is positioned adjacent to an appropriate promoter for expression in an eye or ear cell and the promoter and coding sequence are flanked by inverted terminal repeats. The sequence is then further inserted into a vector sequence, which in certain embodiments can replicate in a human cell. The polynucleotide can be an isolated polynucleotide.

In certain embodiments, the vector is a viral expression vector. In certain embodiments, the viral expression vector is an adenoviral vector, an adeno-associated viral (AAV) vector, or a lentiviral vector. In some embodiments, the viral expression vector is a self-complementary adeno-associated viral vector. In certain embodiments, the AAV vector is based on a single serotype of AAV, such as AAV1, AAV2, AAV3, AAV4, AAV5, AAV6, AAV7, AAV8, and AAV9. Modified AAV vectors based substantially on a single serotype are known in the art, for example serotype rh.74 which is AAV8-like and shares 93% amino acid identity with AAV8 can be used (see, e.g., Martin et al., Am. J. Cell. Physiol. 296:C476-C488, 2009, incorporated herein by reference). Alternatively, the adeno-associated viral vector is a chimeric adeno-associated viral vector based on multiple serotypes of AAV.

The expression vectors provided by the instant invention can include any sequence that encodes a functional miR-96, miR-182, and/or miR-183 for use in any of the methods of the instant invention. In certain embodiments, the expression vector includes a nucleic acid sequence selected from the group consisting of SEQ ID NOs: 53-55.

The invention provides cells containing a polynucleotide encoding miR-96, miR-182, and/or miR-183 of the instant invention. In some embodiments, the cell is a bacterial cell or a mammalian cell. The cell can be a cell to which the polynucleotide has been delivered as a therapeutic intervention. Alternatively, the cell can be a cell in vitro used for the preparation of a pharmaceutical composition for the treatment of ophthalmological or otic disorders.

In some embodiments, the invention provides compositions comprising at least one of the miR-96, miR-182, and miR-183 mimetic compounds or pharmaceutically acceptable salts thereof, and pharmaceutically acceptable carriers or excipients. In some other embodiments, the invention provides compositions comprising an expression vector encoding at least one of miR-96, miR-182, and miR-183, and pharmaceutically acceptable carriers or excipients.

The invention further provides methods of treating or preventing ophthalmological conditions such as retinal degeneration or retinitis pigmentosa comprising administering at least one of the miR-96, miR-182, and miR-183 mimetic compounds described herein to a subject in need thereof. The invention also encompasses methods for improving or restoring visual acuity in a subject in need thereof comprising administering at least one of the miR-96, miR-182, and miR-183 mimetic compounds described herein to a subject in need thereof.

The invention further provides methods of treating or preventing ophthalmological conditions such as retinal degeneration or retinitis pigmentosa comprising administering an expression vector encoding at least one of miR-96, miR-182, and miR-183 to a subject in need thereof. The invention also encompasses methods for improving or restoring visual acuity in a subject in need thereof comprising administering an expression vector encoding at least one of miR-96, miR-182, and miR-183 to a subject in need thereof.

### BRIEF DESCRIPTION OF THE DRAWINGS

FIGS. 1A-ID show quantitation of miRNA mimics and Rho siRNA in mouse retina using a sandwich ELISA assay. Values shown are pmol oligo/g of retina tissue and represent 4 retinas (microRNAs) or 2 retinas (siRNA). FIG. 1A shows the amount of miR-183 mimic, FIG. 1B shows the amount of miR-96 mimic, FIG. 1C shows the amount of miR-182 mimic, and FIG. 1D shows the amount of Rho siRNA.

FIG. 2 shows the functional delivery of Rho siRNA to mouse retina and down-regulation of Rho mRNA. The solid line indicates the amount of rhodopsin siRNA detected (nmol/g retina tissue) and the dotted line indicates the fraction of rhodopsin mRNA detected in retina compared to untreated eyes (time 0).

FIGS. 3A-3B show the effect of passive transfection of a miRNA mimic in R-Ret cells. FIG. 3A shows R-Ret cells transfected with 10 μM miR-206 mimic 72 h post-transfection and FIG. 3B shows untreated R-Ret cells.

FIG. 4 shows the results of an adenylate kinase assay of R-Ret cells transfected with miR-206 mimic.

FIG. 5 shows a real time PCR analysis of Rho mRNA in R-Ret cells transfected with various concentrations of cholesterol-conjugated Rho siRNA.

FIGS. 6A-6B show a real time PCR analysis of mRNA expression profile of genes involved in the phototransduction pathway following exposure to 1 μM oligonucleotides. FIG. 6A shows genes that were up-regulated following exposure to 1 μM oligonucleotides. FIG. 6B shows genes that were down-regulated following exposure to 1 μM oligonucleotides.

FIGS. 7A-7B show a real time PCR analysis of mRNA expression profile of genes involved in the phototransduction pathway following exposure to 3 μM oligonucleotide. FIG. 7A shows genes that were up-regulated following exposure to 3 μM oligonucleotides. FIG. 7B shows genes that were down-regulated following exposure to 3 μM oligonucleotides.

FIG. 8 shows a heat map of the log 2-transformed average fold change values of the treatments shown in FIG. 6-7.

FIG. 9 shows the effect of pooled microRNA mimics and Rho siRNA on visual acuity loss in the mouse model of retinitis pigmentosa.

FIG. 10 shows the effect of pooled microRNA mimics and Rho siRNA on visual acuity loss in the mouse model of retinitis pigmentosa.

### DETAILED DESCRIPTION OF THE INVENTION

MicroRNAs (miRNAs) are small, non-protein coding RNAs of about 18 to about 25 nucleotides in length that are derived from individual miRNA genes, from introns of protein coding genes, or from poly-cistronic transcripts that often encode multiple, closely related miRNAs. See review by Carrington et al. (Science, Vol. 301(5631):336-338, 2003). MiRNAs act as repressors of target mRNAs by promoting their degradation or by inhibiting translation.

MiRNAs are transcribed by RNA polymerase II (pol II) or RNA polymerase III (pol III; see Qi et al. (2006) Cellular & Molecular Immunology, Vol. 3:411-419) and arise from initial transcripts, termed primary miRNA transcripts (pri-miRNAs), that are generally several thousand bases long. Pri-miRNAs are processed in the nucleus by the RNase Drosha into about 70- to about 100-nucleotide hairpin-shaped precursors (pre-miRNAs). Following transport to the cytoplasm, the hairpin pre-miRNA is further processed by Dicer to produce a double-stranded miRNA. The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

The microRNA-183/96/182 cluster is expressed in the retina and other sensory organs. This cluster is located on chromosome 6 in mouse, chromosome 7 in human, and chromosome 4 in rat. The sequences for mature miR-96, miR-182, and miR-183 in mouse, human, and rat are given below.

Mature miR-96 sequence in mouse
(SEQ ID NO: 1)
UUUGGCACUAGCACAUUUUUGCU
Mature miR-96 sequence in human
(SEQ ID NO: 2)
UUUGGCACUAGCACAUUUUUGCU
Mature miR-96 sequence in rat
(SEQ ID NO: 3)
UUUGGCACUAGCACAUUUUUGCU
Mature miR-182 sequence in mouse
(SEQ ID NO: 4)
UUUGGCAAUGGUAGAACUCACACCG
Mature miR-182 sequence in human
(SEQ ID NO: 5)
UUUGGCAAUGGUAGAACUCACACU
Mature miR-82 sequence in rat
(SEQ ID NO: 6)
UUUGGCAAUGGUAGAACUCACACCG
Mature miR-183 sequence in mouse
(SEQ ID NO: 7)
UAUGGCACUGGUAGAAUUCACU
Mature miR-183 sequence in human
(SEQ ID NO: 8)
UAUGGCACUGGUAGAAUUCACU
Mature miR-183 sequence in rat
(SEQ ID NO: 9)
UAUGGCACUGGUAGAAUUCACU

A knockout of the microRNA-183/96/182 cluster in mice resulted in the early-onset and progressive synaptic defects of the photoreceptors and progressive retinal degeneration (Lumayag et al., (2013) Proc Nat Acad Sci, 110(6) E507-E516). Although the microRNA-183/96/182 cluster has been shown to play a role in the development of the retina; it is not yet clear whether restoring or supplementing the activity of the microRNA-183/96/182 cluster in adults could ameliorate or prevent retinal disorders.

The present invention is based, in part, on the discovery that the administration of microRNA mimetic compounds that mimic the activity of miR-96, miR-182, and/or miR-183 regulates expression of phototransduction genes in retinal cells, prevents or reduces the death of photoreceptor cells, and prevents or reduces the loss of vision in a mouse model of retinitis pigmentosa. Accordingly, the present invention provides microRNA mimetic compounds, expression vectors encoding miR-96, miR-182, and/or miR-183, compositions thereof, and methods thereof, for treating or preventing ophthalmological conditions. The invention also contemplates the use of miR-96, miR-182, and/or miR-183 mimetic compounds and expression vectors encoding miR-96, miR-182, and/or miR-183 for treating, improving, or preventing diseases or disorders of other sensory organs, for example, ear.

A microRNA mimetic compound according to the invention comprises a first strand and a second strand, wherein the first strand comprises a mature miR-96, miR-182, or miR-183 sequence and the second strand comprises a sequence that is substantially complementary to the first strand and has at least one modified nucleotide, wherein the microRNA mimetic compound mimics the activity of miR-96, miR-182, or miR-183 microRNA. The term “microRNA agonist” as used herein refers to a synthetic microRNA mimetic compound or an expression vector encoding miR-96, miR-182, and/or miR-183. Throughout the disclosure, the term “first strand” may be used interchangeably with the term “antisense strand” or “guide strand”, and the term “second strand” may be used interchangeably with the term “sense strand” or “passenger strand.”

Synthetic microRNA Mimetic Compounds

In one embodiment, the first strand of the microRNA mimetic compound comprises from about 20 to about 28 nucleotides and the second strand comprises from about 18 to about 26 nucleotides. In various embodiments, the first strand may comprise about 18, 19, 20, 21, 22, 23, 24, 25, 26, 27, or 28 nucleotides and the second strand may comprise about 20, 21, 22, 23, 24, 25, or 26 nucleotides. In certain embodiments, the first strand comprises from about 22 to about 26 nucleotides comprising a sequence of mature miR-96, miR-182, or miR-183, and the second strand comprises from about 20 to about 24 nucleotides comprising a sequence that is fully or substantially complementary to the first strand. In other embodiments, the first strand comprises from about 22 to about 26 nucleotides comprising a sequence of mature miR-96, miR-182, or miR-183, and the second strand comprises from about 20 to about 26 nucleotides comprising a sequence that is fully or substantially complementary to the first strand.

The nucleotides that form the first and the second strand of the microRNA mimetic compounds may comprise ribonucleotides, deoxyribonucleotides, modified nucleotides, and combinations thereof. In certain embodiments, the first strand and the second strand of the microRNA mimetic compound comprise ribonucleotides and/or modified ribonucleotides. The term “modified nucleotide” means a nucleotide where the nucleobase and/or the sugar moiety is modified relative to unmodified nucleotides.

In certain embodiments, the microRNA mimetic compounds have a first strand or an antisense strand comprising a “miRNA region” whose sequence is identical to all or part of a mature miR-96, miR-182, or miR-183 sequence, and a second strand or a sense strand having a “complementary region” whose sequence is from between about 70% to about 100% complementary to the sequence of the miRNA region. The term “miRNA region” refers to a region on the first strand of the miRNA mimetic compound that is at least about 75, 80, 85, 90, 95, or 100% identical, including all integers there between, to the entire sequence of a mature, naturally occurring miR-96, miR-182, or miR-183 sequence. In certain embodiments, the miRNA region is about or is at least about 90, 91, 92, 93, 94, 95, 96, 97, 98, 99, or 100% identical to the sequence of a mature, naturally-occurring miRNA, such as the mouse, human, or rat miR-96, miR-182, or miR-183 sequence. For example, in some embodiments, the miRNA region is about or at least about 99, 99.1, 99.2, 99.3, 99.4, 99.5, 99.6, 99.7, 99.8, 99.9 or 100% identical to the sequence of a mature, naturally-occurring miRNA, such as the mouse, human, or rat miR-96, miR-182, or miR-183 sequence. Alternatively, the miRNA region can comprise 18, 19, 20, 21, 22, 23, 24, 25 or more nucleotide positions in common with a mature, naturally-occurring miRNA as compared by sequence alignment algorithms and methods well known in the art. It is understood that the sequence of the miRNA region of the first strand may include modifications of the nucleotides compared to the sequence of a mature, naturally-occurring miRNA. For example, if a mature, naturally-occurring miRNA sequence comprises a cytidine nucleotide at a specific position, the miRNA region of the first strand of the mimetic compound may comprise a modified cytidine nucleotide, such as 2′-fluoro-cytidine, at the corresponding position or if a mature, naturally-occurring miRNA sequence comprises a uridine nucleotide at a specific position, the miRNA region of the first strand of the mimetic compound may comprise a modified uridine nucleotide, such as 2′-fluoro-uridine, 2′-O-methyl-uridine, 5-fluorouracil, or 4-thiouracil at the corresponding position. Even if the sequence of the miRNA region of the first strand includes such modified nucleotides, the sequence is still considered identical to the sequence of the mature, naturally-occurring miRNA sequence as long as the nucleotide that is modified has the same base-pairing capability as the nucleotide present in the mature, naturally-occurring miRNA sequence. In some embodiments, the first strand may include a modification of the 5′-terminal residue. For example, the first strand may have a 5′-terminal monophosphate.

The term “complementary region” refers to a region on the second strand of the miRNA mimetic compound that is at least about 70% complementary to the sequence of the miRNA region on the first strand. For example, the complementary region is at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, inclusive of all values therebetween, complementary to the sequence of the miRNA region. In certain embodiments, about 18, 19, 20, 21, 22, or 23 nucleotides of the complementary region of the second strand may be complementary to the first strand. In some embodiments, the complementary region of the second strand comprises about 1, 2, 3, 4, or 5 mismatches relative to the miRNA region of the first strand. That is, up to 1, 2, 3, 4, or 5 nucleotides between the miRNA region of the first strand and the complementary region of the second strand may not be complementary. In one embodiment, the mismatches are consecutive and may create a bulge. In another embodiment, the mismatches are not consecutive and may be distributed throughout the complementary region. In yet another embodiment, up to 1, 2, 3, or 4 mismatches may be consecutive creating a bulge and the remaining mismatches may be distributed through the complementary region.

In some embodiments, the first and/or the second strand of the mimetic compound may comprise an overhang on the 5′ or 3′ end of the strands. In certain embodiments, the first strand comprises a 3′ overhang, i.e., a single-stranded region that extends beyond the duplex region, relative to the second strand. In some embodiments, the second strand comprises a 3′ overhang relative to the first strand. The 3′ overhang of the first or the second strand may range from about one nucleotide to about four nucleotides. In certain embodiments, the 3′ overhang of the first or the second strand may comprise 1 or 2 nucleotides. In some embodiments, the nucleotides comprising the 3′ overhang are linked by phosphorothioate linkages. The nucleotides comprising the 3′ overhang may include ribonucleotides, deoxyribonucleotides, modified nucleotides, or combinations thereof. In certain embodiments, the 3′ overhang in the first or the second strand comprises two ribonucleotides. In other embodiments, the 3′ overhang in the first or the second strand comprises two modified ribonucleotides. In still other embodiments, the 3′ overhang in the first or the second strand comprises one ribonucleotide and one modified ribonucleotide. In some embodiments, the overhang may be present on the 5′ end of the first or the second strand. The 5′ overhand may comprise from about one to four nucleotides. Similar to the 3′ overhang, the 5′ overhang may comprise ribonucleotides, deoxyribonucleotides, modified nucleotides, or combinations thereof. In some embodiments, the nucleotides comprising the 5′ overhang may be linked by phosphorothioate linkages. In some embodiments, the miRNA mimetic compound may be a hairpin, i.e., a single strand polynucleotide with a 5′ and a 3′ end, where one of the ends may generate an overhang when the single strand folds back on itself. In these embodiments, the single strand comprises the miRNA region and the complementary region that may be separated by a linker region. Such a single strand miRNA mimetic compound may have a greater range of length, for example, about 55 to about 100 nucleotides. The single stranded miRNA mimetic compound may contain an unpaired loop that would substantially correspond to the linker region.

It will be understood from the above description that the first or the antisense strand of the microRNA mimetic compound may comprise the entire sequence of a mature, naturally occurring microRNA or a part of it and may comprise additional nucleotides that are not part of the mature miRNA sequence. For example, the first strand of a mimetic compound according to the invention that mimics the activity of miR-96 may comprise the entire sequence of SEQ ID NOs: 1, 2, or 3 or a partial sequence, such as about, 15, 16, 17, 18, 19, 20, 21, or 22 nucleotides of SEQ ID NOs: 1, 2, or 3 and up to about 4 to 6 additional nucleotides that are not part of the mature miR-96 sequence. It will also be understood that the mimetic compound comprising the entire or partial sequence of the mature, naturally occurring microRNA and up to about 4 to 6 additional nucleotides still maintains the ability to mimic the activity or function of the microRNA. It will also be understood that the sequence of the first strand comprising the sequence of a mature, naturally occurring microRNA may include modified nucleotides corresponding to the nucleotides present in the mature, naturally-occurring microRNA.

In one embodiment, the invention provides a microRNA mimetic compound comprising a first strand of about 22 to about 26 ribonucleotides comprising a mature miR-96, miR-182, or miR-183 sequence; and a second strand of about 20 to about 24 ribonucleotides comprising a sequence that is substantially complementary to the first strand and having at least one modified nucleotide, wherein the first strand has a 3′ nucleotide overhang relative to the second strand. In some embodiments, the invention provides a microRNA mimetic compound comprising a first strand of about 22 to about 26 ribonucleotides comprising a mature miR-96, miR-182, or miR-183 sequence; and a second strand of about 20 to about 26 ribonucleotides comprising a sequence that is substantially complementary to the first strand and having at least one modified nucleotide, wherein the second strand has a 3′ nucleotide overhang relative to the second strand. The term “substantially complementary” means the sequence of the second strand is at least about 75, 76, 77, 78, 79, 80, 81, 82, 83, 84, 85, 86, 87, 88, 89, 90, 91, 92, 93, 94, 95, 96, 97, 98, 99, or 100%, inclusive of all values therebetween, complementary to the sequence of the first strand or the sequence of the second strand contains up to about 1, 2, 3, 4, 5, or 6 mismatches relative to the sequence of the first strand.

In certain embodiments, a miR-96 mimetic compound according to the invention comprises a first strand of about 22 to about 26 nucleotides comprising a sequence of SEQ ID NOs: 1, 2, or 3, and a second strand of about 20 to about 24 nucleotides that is substantially complementary to the first strand and having at least one modified nucleotide, wherein the first strand has a 3′ nucleotide overhang relative to the second strand. In some embodiments, the second strand may comprise about 20 to about 26 nucleotides and may include a 3′ or 5′ nucleotide overhang relative to the first strand. Throughout this disclosure, the language reciting “a sequence of SEQ ID NOs. X, Y. or Z” encompasses all sequences where a naturally occurring nucleotide is replaced by a corresponding modified nucleotide. For example, the language encompasses sequences where adenosine is replaced with a modified adenosine; uridine is replaced with a modified uridine, thymidine, or modified thymidine; guanosine is replaced with modified guanosine; or cytidine is replaced with modified cytidine.

In certain embodiments, a miR-96 mimetic compound according to the invention comprises a first strand comprising the sequence: 5′-rUrUfUrGrGfCrAfCfUrArGfCrAfCrAfUfUfUfUfUrGfCfUsrUsrU-3′ (SEQ ID NO: 10) and a second strand that is substantially complementary, such as about 85, 86, 87, 88, 89, 90, 91, 92, 93, 94, 95, 96, 97, 98, 99, or 100%, inclusive of all values therebetween, complementary to the first strand. In one embodiment, the second strand of a miR-96 mimetic compound comprises the sequence: 5′-mAmGmCrArArArAmUmUrGrArGmCmUrGmCrGrArArA-3′ (SEQ ID NO: 11). In another embodiment, the second strand of a miR-96 mimetic compound comprises the sequence: 5′-mAmGmCrArArArArAmUrGmUrGmCmUrArGmUrGmCmCrArArA-3′ (SEQ ID NO: 12) As used herein, an “m” preceding a base notation (e.g. A, U, G. C) indicates a 2′-O-methyl modified nucleotide, an “r” preceding a base notation indicates an unmodified ribonucleotide (i.e., 2′-OH), an “f” preceding a base notation indicates a 2′-fluoronucleotide, and an “s” indicates a phosphorothioate linkage. Unless otherwise indicated, the nucleotides in the antisense and the sense strand are linked by phosphodiester linkages. In some embodiments, a miR-96 mimetic compound according to the invention comprises a first strand comprising a sequence selected from SEQ ID NOs: 10 and 26-29. In some embodiments, a miR-96 mimetic compound according to the invention comprises a second strand comprising a sequence selected from SEQ ID NOs: 11-14 and 30-34.

In certain embodiments, a miR-96 mimetic compound according to the invention comprises a first strand of about 22 to about 26 ribonucleotides comprising a sequence of SEQ ID NOs: 1, 2, 3, or 10 and a second strand of about 20 to about 24 ribonucleotides comprising a sequence that is substantially complementary to the first strand and having at least one modified nucleotide, wherein the first strand has a 3′ nucleotide overhang relative to the second strand. In certain embodiments, the second strand of a miR-96 mimetic compound comprises a sequence of SEQ ID NO: 11 or 12. In some embodiments, a miR-96 mimetic compound according to the invention comprises a first strand of about 22 to about 26 ribonucleotides comprising a sequence of SEQ ID NOs: 1, 2, 3, or 10 and a second strand of about 20 to about 26 ribonucleotides comprising a sequence that is substantially complementary to the first strand and having at least one modified nucleotide, wherein the second strand has a 3′ or 5′ nucleotide overhang relative to the first strand.

In some embodiments, a miR-96 mimetic compound comprises a first strand of about 20 to about 26 nucleotides comprising the sequence of SEQ ID NO: 10 and a second strand of about 20 to about 24 nucleotides comprising the sequence of SEQ ID NO: 11, wherein the first strand has a 3′ overhang relative to the second strand. In another embodiment, a miR-96 mimetic compound comprises a first strand of about 20 to about 26 nucleotides comprising the sequence of SEQ ID NO: 10 and a second strand of about 20 to about 24 nucleotides comprising the sequence of SEQ ID NO: 12, wherein the first strand has a 3′ overhang relative to the second strand. In yet another embodiment, a miR-96 mimetic compound comprises a first strand of about 20 to about 26 nucleotides comprising the sequence of SEQ ID NO: 10 and a second strand of about 20 to about 24 nucleotides comprising the sequence of SEQ ID NO: 13 (5′-mA.mG.mC.rA.rA.rA.rA.mU.mU.rG.rA.rG.mC.mU.rA.rG.mU.rG.mC.rG.rA.rA.rA.cho16-3′), wherein the first strand has a 3′ overhang relative to the second strand. In some embodiments, the second strand of a miR-96 mimetic compound is about 20 to about 24 nucleotides and comprises the sequence of SEQ ID NO: 14 (5′-mA.mG.mC.rA.rA.rA.rA.mU.mU.rG.rA.rG.mC.mU.rA.rG.mU.rG.mC.rG.rA.rA.rAs.cho16-3′). In this embodiment, the oligonucleotide sequence of the second strand is attached to cholesterol (carrier molecule) via a phosphorothioate linkage.

In some embodiments, a miR-96 mimetic compound comprises a first strand that is no more than 25, 26, 27 or 28 nucleotides long and comprises a sequence of SEQ ID NOs: 1, 2, 3, or 10. In some other embodiments, a miR-96 mimetic compound comprises a first strand that is no more than 26, 27 or 28 nucleotides long and comprises a sequence of SEQ ID NOs: 1, 2, or 3, where nucleotides at positions 3(U), 6(C), 8(C), 9(U), 12 (C), 14 (C), 16 (U), 17 (U), 18 (U), 19 (U), 20 (U), 22 (C), and/or 23 (U) in the 5′ to 3′ direction are modified relative to SEQ ID NOs. 1, 2, or 3. In certain embodiments, a miR-96 mimetic compound comprises a first strand having a sequence of SEQ ID NOs: 1, 2, 3, or 10, and a second strand that is complementary to the first strand except at nucleotide positions 4, 13, and/or 16 from the 3′ end of the second strand. In some embodiments, the second strand of a miR-96 mimetic compound is complementary to the sequence of SEQ ID NOs: 1, 2, 3, or 10 and comprises modified nucleotides at one ore more positions selected from the group consisting of 1(A), 2(G), 3(C), 8(U), 9(U), 13(C), 14(U), 17(U), and 19(C), in the 5′ to 3′ direction.

In certain embodiments, a miR-182 mimetic compound according to the invention comprises a first strand of about 22 to about 26 nucleotides comprising a sequence of SEQ ID NOs: 4, 5, or 6, and a second strand that is substantially complementary to the first strand and having at least one modified nucleotide, wherein the first strand has a 3′ nucleotide overhang relative to the second strand. In some embodiments, the second strand may comprise about 20 to about 26 nucleotides and may include a 3′ or 5′ nucleotide overhang relative to the first strand. In certain embodiments, a miR-182 mimetic compound according to the invention comprises a first strand comprising the sequence: 5′-rUrUfUrGrGfCrArAfUrGrGfUrArGrArAfCfUfrAfCrAfUsrUsrU-3′ (SEQ ID NO: 15) and a second strand that is substantially complementary, such as about 85, 86, 87, 88, 89, 90, 91, 92, 93, 94, 95, 96, 97, 98, 99, or 100%, inclusive of all values therebetween, complementary to the first strand. In one embodiment, the second strand of a miR-182 mimetic compound comprises the sequence: 5′-mAmGmUrGmUrGrArGrAmUmCrArAmCmCrAmUmUrGmCrGrArArA-3′ (SEQ ID NO: 16). In another embodiment, the second strand of a miR-182 mimetic compound comprises the sequence: 5′-mAmGmUrGmUrGrArGmUmUmCmUrAmCmCrAmUmUrGmCmCrArArA-3′ (SEQ ID NO: 17). In some embodiments, a miR-182 mimetic compound according to the invention comprises a first strand comprising a sequence selected from SEQ ID NOs: 15 and 35-38. In some embodiments, a miR-182 mimetic compound according to the invention comprises a second strand comprising a sequence selected from SEQ ID NOs: 16-19 and 39-43.

In some embodiments, a miR-182 mimetic compound comprises a first strand of about 22 to about 26 ribonucleotides containing a sequence of SEQ ID NOs: 4, 5, 6, or 15 and a second strand of about 20 to about 24 ribonucleotides comprising a sequence that is substantially complementary to the first strand and having at least one modified nucleotide, wherein the first strand has a 3′ nucleotide overhang relative to the second strand. In certain embodiments, the second strand of a miR-182 mimetic compound comprises a sequence of SEQ ID NO: 16 or 17. In some embodiments, a miR-182 mimetic compound according to the invention comprises a first strand of about 22 to about 26 ribonucleotides comprising a sequence of SEQ ID NOs: 4, 5, 6, or 15 and a second strand of about 20 to about 26 ribonucleotides comprising a sequence that is substantially complementary to the first strand and having at least one modified nucleotide, wherein the second strand has a 3′ or 5′ nucleotide overhang relative to the first strand.

In some embodiments, a miR-182 mimetic compound comprises a first strand of about 22 to about 26 nucleotides comprising the sequence of SEQ ID NO: 15 and a second strand of about 20 to about 24 nucleotides comprising the sequence of SEQ ID NO: 16, wherein the first strand has a 3′ overhang relative to the second strand. In another embodiment, a miR-182 mimetic compound comprises a first strand of about 22 to about 26 nucleotides comprising the sequence of SEQ ID NO: 15 and a second strand of about 20 to about 24 nucleotides comprising the sequence of SEQ ID NO: 17, wherein the first strand has a 3′ overhang relative to the second strand. In yet another embodiment, a miR-182 mimetic compound comprises a first strand of about 22 to about 26 nucleotides comprising the sequence of SEQ ID NO: 15 and a second strand of about 20 to about 24 nucleotides comprising the sequence of SEQ ID NO: 18 (5′-mA.mG.mU.rG.mU.rG.rA.rG.rA.mU.mC.rA.rA.mC.mC.rA.mU.mU.rG.mC.rG.rA.rA.rA.cho 16-3′), wherein the first strand has a 3′ overhang relative to the second strand. In some embodiments, the second strand of a miR-182 mimetic compound is about 20 to about 24 nucleotides and comprises the sequence of SEQ ID NO: 19 (5′-mA.mG.mU.rG.mU.rG.rA.rG.rA.mU.mC.rA.rA.mC.rA.mU.mU.rG.mC.rG.rA.rA.rAs.cho16-3′). In this embodiment, the oligonucleotide sequence of the second strand is attached to cholesterol (carrier molecule) via a phosphorothioate linkage.

In some embodiments, a miR-182 mimetic compound comprises a first strand that is no more than 26, 27 or 28 nucleotides long and comprises a sequence of SEQ ID NOs: 4, 5, 6, or 15. In some other embodiments, a miR-182 mimetic compound comprises a first strand that is no more than 27 or 28 nucleotides long and comprises a sequence of SEQ ID NOs: 4, 5, or 6, where nucleotides at positions 3(U), 6(C), 9(U), 12 (U), 17 (C), 18 (U), 19 (C), 21 (C), 23 (C), and/or 24 (U) in the 5′ to 3′ direction are modified relative to SEQ ID NOs. 4, 5, or 6. In certain embodiments, a miR-182 mimetic compound comprises a first strand having a sequence of SEQ ID NOs: 4, 5, 6, or 15, and a second strand that is complementary to the first strand except at nucleotide positions 4, 13, and/or 16 from the 3′ end of the second strand. In some embodiments, the second strand of a miR-182 mimetic compound is complementary to the sequence of SEQ ID NOs: 4, 5, 6, or 15 and comprises modified nucleotides at one ore more positions selected from the group consisting of 1(A), 2 (G), 3(U), 5(U), 10(U), 11(C), 14(C), 15(C), 17(U), 18(U), and 20(C), in the 5′ to 3′ direction.

In certain embodiments, a miR-183 mimetic compound according to the invention comprises a first strand of about 22 to about 26 nucleotides comprising a sequence of SEQ ID NOs: 7, 8, or 9, and a second strand that is substantially complementary to the first strand and having at least one modified nucleotide, wherein the first strand has a 3′ nucleotide overhang relative to the second strand. In some embodiments, the second strand may comprise about 20 to about 26 nucleotides and may include a 3′ or 5′ nucleotide overhang relative to the first strand. In certain embodiments, a miR-183 mimetic compound according to the invention comprises a first strand comprising the sequence: 5′-rUrAfUrGrGfCrAfCfUrGrGfUrArGrArAfUfUfCrAfCfUsrUsrU-3′ (SEQ ID NO: 20) and a second strand that is substantially complementary, such as about 85, 86, 87, 88, 89, 90, 91, 92, 93, 94, 95, 96, 97, 98, 99, or 100%, inclusive of all values therebetween, complementary to the first strand. In one embodiment, the second strand of a miR-183 mimetic compound comprises the sequence: 5′-mAmGmUrGrArArAmUmCrArAmCmCrArGmUrGmCrGrAmUrA-3′ (SEQ ID NO: 21). In another embodiment, the second strand of a miR-183 mimetic compound comprises the sequence: 5′-mAmGmUrGrArAmUmUmCmUrAmCmCrArGmUrGmCmrAmUrA-3′ (SEQ ID NO: 22). In some embodiments, a miR-183 mimetic compound according to the invention comprises a first strand comprising a sequence selected from SEQ ID NOs: 20 and 44-47. In some embodiments, a miR-183 mimetic compound according to the invention comprises a second strand comprising a sequence selected from SEQ ID NOs: 21-25 and 48-52.

In some embodiments, a miR-183 mimetic compound comprises a first strand of about 22 to about 26 ribonucleotides comprising a sequence of SEQ ID NOs: 7, 8, 9, or 20 and a second strand of about 20 to about 24 ribonucleotides comprising a sequence that is substantially complementary to the first strand and having at least one modified nucleotide, wherein the first strand has a 3′ nucleotide overhang relative to the second strand. In certain embodiments, the second strand of a miR-183 mimetic compound comprises a sequence of SEQ ID NO: 21 or 22. In some embodiments, a miR-183 mimetic compound according to the invention comprises a first strand of about 22 to about 26 ribonucleotides comprising a sequence of SEQ ID NOs: 7, 8, 9, or 20 and a second strand of about 20 to about 26 ribonucleotides comprising a sequence that is substantially complementary to the first strand and having at least one modified nucleotide, wherein the second strand has a 3′ or 5′ nucleotide overhang relative to the first strand.

In some embodiments, a miR-183 mimetic compound comprises a first strand of about 22 to about 26 nucleotides comprising the sequence of SEQ ID NO: 20 and a second strand of about 18 to about 22 nucleotides comprising the sequence of SEQ ID NO: 21, wherein the first strand has a 3′ overhang relative to the second strand. In another embodiment, a miR-183 mimetic compound comprises a first strand of about 22 to about 26 nucleotides comprising the sequence of SEQ ID NO: 20 and a second strand of about 18 to about 22 nucleotides comprising the sequence of SEQ ID NO: 22, wherein the first strand has a 3′ overhang relative to the second strand. In yet another embodiment, a miR-183 mimetic compound comprises a first strand of about 22 to about 26 nucleotides comprising the sequence of SEQ ID NO: 20 and a second strand of about 18 to about 22 nucleotides comprising the sequence of SEQ ID NO: 23 (5′-mAmGmUrGrArArAmUmCrArAmCmCrArGmUrGmCrGrAmUrA.cho16-3′) or SEQ ID NO: 24 (5′-mAmGmUrGrArAmUmUmCmUrAmCmCrArGmUrGmCmCrAmUrA.cho16-3′), wherein the first strand has a 3′ overhang relative to the second strand. In one embodiment, the second strand of a miR-183 mimetic compound comprises the sequence of SEQ ID NO: 25 (5′-mAmGmUrGrArArAmUmCrArAmCmCrArGmUrGmCrGrAmUrAs.cho16-3′). In this embodiment, the oligonucleotide sequence of the second strand is attached to cholesterol (carrier molecule) via a phosphorothioate linkage.

In some embodiments, a miR-183 mimetic compound comprises a first strand that is no more than 25, 26, 27 or 28 nucleotides long and comprises a sequence of SEQ ID NOs: 7, 8, 9, or 20. In some other embodiments, a miR-183 mimetic compound comprises a first strand that is no more than 25, 26, 27 or 28 nucleotides long and comprises a sequence of SEQ ID NOs: 7, 8, or 9, where nucleotides at positions 3(U), 6(C), 8(C), 9(U), 12(U), 17(U), 18(U), 19(C), 21(C), and/or 22(U) in the 5′ to 3′ direction are modified relative to SEQ ID NOs. 7, 8, or 9. In certain embodiments, a miR-183 mimetic compound comprises a first strand having a sequence of SEQ ID NOs: 7, 8, 9, or 20, and a second strand that is complementary to the first strand except at nucleotide positions 4, 13, and/or 16 from the 3′ end of the second strand. In some embodiments, the second strand of a miR-183 mimetic compound is complementary to the sequence of SEQ ID NOs: 7, 8, 9, or 20 and comprises modified nucleotides at one ore more positions selected from the group consisting of 1(A), 2(G), 3(U), 7(U), 8(U), 9(C), 10(U), 12(C), 13(C), 16(U), 18(C), 19(C), and 21(U), in the 5′ to 3′ direction.

Specific microRNA mimetic compounds disclosed herein are summarized in Table 1 below. However, the invention is not limited to these specific mimetic compounds and other mimetic compounds that could be prepared based on the guidance provided throughout the specification are also encompassed by the invention.

TABLE 1
miR-96/182/183 mimics

| | |
|---|---|
| SEQ | |
| ID NO. | Modified Sequence |

miR-96 First/antisense/guide strands

| | |
|---|---|
| 10 | 5′-rUrUfUrGrGfCrAfCfUrArGfCrAfCrAfUfUfUfUfUrGfCfUsrUsrU-sup-3′ |
| 26 | 5′-rUrUfUrGrGfCrAfCfUrArGfCrAfCrAfUfUfUfUfUrGfCfU-3′ |
| 27 | 5′-p.rUrUfUrGrGfCrAfCfUrArGfCrAfCrAfUfUfUfUfUrGfCfUsrUsrU-3′ |
| 28 | 5′-rUrUrUrGrGrCrArCrUrArGrCrArCrArUrUrUrUrUrGrCrUrUrU-3′ |
| 29 | 5′-rUrUmUrGrGmCrAmCmUrArGmCrAmCrAmUmUmUmUmUrGmCmUsrUsrU-3′ |

miR-96 Second/sense/passenger strands

| | |
|---|---|
| 11 | 5′-mAmGmCrArArArAmUmUrGrArGmCmUrArGmUrGmCrGrArArA-3′ |
| 12 | 5′-mAmGmCrArArArArAmUrGmUrGmCmUrArGmUrGmCmCrArArA-3′ |
| 13 | 5′-mAmGmCrArArArAmUmUrGrArGmCmUrArGmUrGmCrGrArArAchol6-3′ |
| 14 | 5′-mAmGmCrArArArAmUmUrGrArGmCmUrArGmUrGmCrGrArArAschol6-3′ |
| 30 | 5′-mAmGmCrArArArAmUmUrGrArGmCmUrArGmUrGmCrGrArArAsrUsrU-3′ |
| 31 | 5′-C6Chol.mAmGmCrArArArAmUmUrGrArGmCmUrArGmUrGmCrGrArArA-3′ |
| 32 | 5′-mAmGrCrArArArArUrGrUrGrCrUrArGrUrGrCrCrArArArUrU-3′ |
| 33 | 5′-mAmGmCrArArArAmUmUrGrArGmCmUrArGmUrGmCrGrArArAsChol6-3′ |
| 34 | 5′-mAmGmCrArArArAmUmUrGrArGmCmUrArGmUrGmCrGrArArAChol6-3′ |

miR-182 First/antisense/guide strands

| | |
|---|---|
| 15 | 5′-rU.rU.fU.rG.rG.fC.rA.rA.fU.rG.rG.fU.rA.rG.rA.rA.fC.fU.fC.rA.fC.rA.fC.fUs.rUs.rU-sup-3′ |
| 35 | 5′-rUrUfUrGrGfCrArAfUrGrGfUrArGrArAfCfUfCrAfCrAfCfU-3′ |
| 36 | 5′-p.rUrUfUrGrGfCrArAfUrGrGfUrArGrArAfCfUfCrAfCrAfCfUsrUsrU-3′ |
| 37 | 5′-rUrUrUrGrGrCrArArUrGrGrUrArGrArArCrUrCrArCrArCrUrUrU-3′ |
| 38 | 5′-rUrUmUrGrGmCrArAmUrGrGmUrArGrArAmCmUmCrAmCrAmCmUsrUsrU-3′ |

miR-182 Second/sense/passenger strands

| | |
|---|---|
| 16 | 5′-mA.mG.mU.rG.mU.rG.rA.rG.rA.mU.mC.rA.rA.mC.mC.rA.mU.mU.rG.mC.rG.rA.rA.rA-3′ |
| 17 | 5′-mA.mG.mU.rG.mU.rG.rA.rG.mU.mU.mC.mU.rA.mC.mC.rA.mU.mU.rG.mC.mC.rA.rA.rA.-3′ |
| 18 | 5′-mA.mG.mU.rG.mU.rG.rA.rG.rA.mU.mC.rA.rA.mC.mC.rA.mU.mU.rG.mC.rG.rA.rA.rA.chol6-3′ |
| 19 | 5′-mA.mG.mU.rG.mU.rG.rA.rG.rA.mU.mC.rA.rA.mC.mC.rA.mU.mU.rG.mC.rG.rA.rA.rAs.chol6-3′ |
| 39 | 5′-mAmGmUrGmUrGrArGrAmUmCrArAmCmCrAmUmUrGmCrGrArArAsrUsrU-3′ |
| 40 | 5′-C6chol.mAmGmUrGmUrGrArGrAmUmCrArAmCmCrAmUmUrGmCrGrArArA-3′ |
| 41 | 5′-mAmGrUrGrUrGrArGrUrUrCrUrArCrCrArUrUrGrCrCrArArArUrU-3′ |
| 42 | 5′-mAmGmUrGmUrGrArGrAmUmCrArAmCmCrAmUmUrGmCrGrArArAsChol6-3′ |
| 43 | 5′-mAmGmUrGmUrGrArGrAmUmCrArAmCmCrAmUmUrGmCrGrArArAChol6-3′ |

miR-183 First/antisense/guide strands

| | |
|---|---|
| 20 | 5′-rU.rA.fU.rG.rG.fC.rA.fC.fU.rG.rG.fU.rA.rG.rA.rA.fU.fU.fC.rA.fC.fUs.rUs.rU-sup-3′ |
| 44 | 5′-rUrAfUrGrGfCrAfCfUrGrGfUrArGrArAfUfUfCrAfCfU-3′ |
| 45 | 5′-p.rUrAfUrGrGfCrAfCfUrGrGfUrArGrArAfUfUfCrAfCfUsrUsrU-3′ |
| 46 | 5′-rUrArUrGrGrCrArCrUrGrGrUrArGrArArUrUrCrArCrUrUrU-3′ |
| 47 | 5′-rUrAmUrGrGmCrAmCmUrGrGmUrArGrArAmUmUmCrAmCmUsrUsrU-3′ |

miR-183 Second/sense/passenger strands

| | |
|---|---|
| 21 | 5′-mA.mG.mU.rG.rA.rA.rA.mU.mC.rA.rA.mC.mC.rA.rG.mU.rG.mC.rG.rA.mU.rA-3′ |
| 22 | 5′-mA.mG.mU.rG.rA.rA.mU.mU.mC.mU.rA.mC.mC.rA.rG.mU.rG.mC.mC.rA.mU.rA.-3′ |
| 23 | 5′-mA.mG.mU.rG.rA.rA.rA.mU.mC.rA.rA.mC.mC.rA.rG.mU.rG.mC.rG.rA.mU.rA.chol6-3′ |
| 24 | 5′-mA.mG.mU.rG.rA.rA.mU.mU.mC.mU.rA.mC.mC.rA.rG.mU.rG.mC.mC.rA.mU.rA.chol6-3′ |
| 25 | 5′-mA.mG.mU.rG.rA.rA.rA.mU.mC.rA.rA.mC.mC.rA.rG.mU.rG.mC.rG.rA.mU.Ras.chol6-3′ |
| 48 | 5′-mAmGmUrGrArAmUmUmCmUrAmCmCrArGmUrGmCmCrAmUrAsrUsrU-3′ |
| 49 | 5′-C6chol.mAmGmUrGrArArAmUmCrArAmCmCrArGmUrGmCrGrAmUrA-3′ |
| 50 | 5′-mAmGrUrGrArArUrUrCrUrArCrCrArGrUrGrCrCrArUrArUrU-3′ |
| 51 | 5′-mAmGmUrGrArArAmUmCrArAmCmCrArGmUrGmCrGrAmUrAChol6-3′ |
| 52 | 5′-mAmGmUrGrArArAmUmCrArAmCmCrArGmUrGmCrGrAmUrAsChol6-3; |

The modified nucleotides that may be used in the microRNA mimetic compounds of the invention can include nucleotides with a base modification or substitution. The natural or unmodified bases in RNA are the purine bases adenine (A) and guanine (G), and the pyrimidine bases cytosine (C) and uracil (U) (DNA has thymine (T)). In contrast, modified bases, also referred to as heterocyclic base moieties, include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In some embodiments, the microRNA mimetic compounds can have nucleotides with modified sugar moieties. Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an RNA molecule may have one or more nucleotides with a sugar modification at one position and also one or more nucleotides with a sugar modification at a different position.

Sugar modifications contemplated in the miRNA mimetic compounds include, but are not limited to, a substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments, these groups may be chosen from: O(CH₂)ₓOCH₃, O((CH₂)ₓO)_{y}CH₃, O(CH₂)ₓNH₂, O(CH₂)ₓCH₃, O(CH₂)ₓONH₂, and O(CH₂)ₓON((CH₂)ₓCH₃)₂, where x and y are from 1 to 10.

In some embodiments, miRNA mimetic compounds have a sugar substituent group selected from the following: C₁ to C₁₀ lower alkyl, substituted lower alkyl, alkenyl, alkynyl, alkaryl, aralkyl, O-alkaryl or O-aralkyl, SH, SCH₃, Cl, Br, CN, OCN, CF₃, OCF₃, SOCH₃, SO₂CH₃, ONO₂, NO₂, N₃, NH₂, heterocycloalkyl, heterocycloalkaryl, aminoalkylamino, or similar substituents. In one embodiment, the modification includes 2′-methoxyethoxy (2′-O—CH₂CH₂OCH₃, which is also known as 2′-O-(2-methoxyethyl) or 2′-MOE), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Additional sugar substituent groups include allyl (—CH₂—CH═CH₂), —O-allyl, methoxy (—O—CH₃), aminopropoxy (—OCH₂CH₂CH₂NH₂), and fluoro (F). Sugar substituent groups on the 2′ position (2′-) may be in the arabino (up) position or ribo (down) position. One 2′-arabino modification is 2′-F. Other similar modifications may also be made at other positions on the sugar moiety, particularly the 3′ position of the sugar on the 3′ terminal nucleoside or in 2′-5′ linked oligonucleotides and the 5′ position of 5′ terminal nucleotide.

In certain embodiments, the sugar modification is a 2′-O-alkyl (e.g. 2′-O-methyl, 2′-O-methoxyethyl), 2′-halo (e.g., 2′-fluoro, 2′-chloro, 2′-bromo), and 4′ thio modifications. For instance, in some embodiments, the first strand of the miR-96, miR-182, or miR-183 mimetic compound comprises one or more 2′ fluoro nucleotides. In another embodiment, the first strand of the mimetic compounds has no modified nucleotides. In yet another embodiment, the second strand of miR-96, miR-182, or miR-183 mimetic compound comprises at least one 2′-O-methyl modified nucleotide.

The first and the second strand of microRNA mimetic compounds of the invention can also include backbone modifications, such as one or more phosphorothioate, morpholino, or phosphonocarboxylate linkages (see, for example, U.S. Pat. Nos. 6,693,187 and 7,067,641, which are herein incorporated by reference in their entireties). For example, in some embodiments, the nucleotides comprising the 3′ overhang in the first strand are linked by phosphorothioate linkages.

In some embodiments, the microRNA mimetic compounds are conjugated to a carrier molecule such as a steroid (cholesterol), a vitamin, a fatty acid, a carbohydrate or glycoside, a peptide, or other small molecule ligand to facilitate in vivo delivery and stability. Preferably, the carrier molecule is attached to the second strand of the microRNA mimetic compound at its 3′ or 5′ end through a linker or a spacer group. In various embodiments, the carrier molecule is cholesterol, a cholesterol derivative, cholic acid or a cholic acid derivative. The use of carrier molecules disclosed in U.S. Pat. No. 7,202,227, which is incorporated by reference herein in its entirety, is also envisioned. In certain embodiments, the carrier molecule is cholesterol and it is attached to the 3′ or 5′ end of the second strand through at least a six carbon linker. In one embodiment, the carrier molecule is attached to the 3′ end of the second strand through a linker. In various embodiments, the linker comprises a substantially linear hydrocarbon moiety. The hydrocarbon moiety may comprise from about 3 to about 15 carbon atoms and may be conjugated to cholesterol through a relatively non-polar group such as an ether or a thioether linkage. In certain embodiments, the hydrocarbon linker/spacer comprises an optionally substituted C2 to C15 saturated or unsaturated hydrocarbon chain (e.g. alkylene or alkenylene). A variety of linker/spacer groups described in U.S. Pre-grant Publication No. 2012/0128761, which is incorporated by reference herein in its entirety, can be used in the present invention.

Expression Vectors Encoding miR-96, miR-182, and/or miR-183

An expression vector comprising at least one gene encoding miR-96, miR-182, and/or miR-183 includes a sufficient portion of the miR-96, miR-182, and/or miR-183 native coding sequence, with or without flanking sequences present in the genomic context of miR-96, miR-182, and/or miR-183, to produce a mature miR-96, miR-182, and/or miR-183 to regulate expression of at least one miR-96, miR-182, and/or miR-183 target. For example, a sufficient portion can include about 21, 22, 23, 24, 25, 26, 27, 28, 29, 30, 35, 40, 45, 50, 55, 60, 65, 70, 75, 80, 85, 90, 95, or 100 nucleotides, including values and ranges thereof. In one embodiment, a sufficient portion contains at least the sequence of the mature miR. In certain embodiments, a sufficient portion includes at least the hairpin sequence of the miR. In certain embodiments, a sufficient portion includes the full length miR. A sufficient portion can be determined using methods routine in the art. It is understood that a sequence encoding a miR-96, miR-182, and/or miR-183 will be complementary to the RNA sequences provided and include Ts rather than U's when the complementary DNA strand.

By “vector” is meant a nucleic acid molecule, for example, a plasmid, cosmid, or bacteriophage, that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, inverted terminal repeats, and enhancers.

In one embodiment, the expression vector is an AAV-based vector system which is an especially attractive platform for regulatory RNA delivery (Franich et al., Mol Ther 16, 947-956, 2008 and McCarty, Mol Ther 16, 1648-1656, 2008). When delivered in viral vectors, miRNAs are continually transcribed, allowing sustained high level expression in target tissues without the need for repeated dosing. Additionally, the use of tissue-specific promoters could restrict this expression to particular cell types of interest even with systemic delivery of the virus. Compared to retroviral delivery systems, DNA viruses such as AAV carry substantially diminished risk of insertional mutagenesis since viral genomes persist primarily as episomes (Schnepp et al., J Virol 77, 3495-3504, 2003). Further, the availability of multiple AAV serotypes allows efficient targeting of many tissues of interest (Gao et al., Proc Natl Acad Sci USA 99, 11854-11859, 2002: McCarty, Mol Ther 16, 1648-1656, 2008). Finally, the general safety of AAV has been well documented, with clinical trials using this platform already underway (Carter. Hum Gene Ther 16, 541-550, 2005; Maguire et al., N Engl J Med 358, 2240-2248, 2008; and Park et al., Front Biosci 13, 2653-2659, 2008). Recent advances in AAV vector technology include a self-complementary genome which enhances therapeutic gene expression and non-human primate AAV serotypes which facilitate efficient transduction following delivery. Due to their small size, regulatory RNAs are especially amenable to AAV-mediated delivery.

The expression vectors provided by the instant invention can include any sequence that encodes a functional miR-96, miR-182, and/or miR-183 for use in any of the methods of the instant invention. In some embodiments, the expression vector includes a nucleic acid sequence encoding partial or the entire sequence of pre-miRNA hairpin. In certain embodiments, the expression vector includes a nucleic acid sequence selected from SEQ ID NOs. 53-55 (Table 2). The nucleic acid sequence encoding a functional miR-96, miR-182, and/or miR-183 could be present as a single cluster or as two or three separate clusters.

TABLE 2

| miR DNA | |
|---|---|
| Sequence | Sequence |
| miR-182 | 5′-GAGCTGCTTGCCTCCCCCCGTTTTTGGCAATGGTAG |
| | AACTCACACTGGTGAGGTAACAGGATCCGGTGGTTCTAG |
| | ACTTGCCAACTATGGGGCGAGGACTCAGCCGGCAC-3′ |
| | (SEQ ID NO: 53) |
| miR-183 | 5′-CCGCAGAGTGTGACTCCTGTTCTGTGTATGGCACTG |
| | GTAGAATTCACTGTGAACAGTCTCAGTCAGTGAATTACC |
| | GAAGGGCCATAAACAGAGCAGAGACAGATCCACGA-3′ |
| | (SEQ ID NO: 54) |
| miR-96 | 5′-TGGCCGATTTTGGCACTAGCACATTTTTGCTTGTGT |
| | CTCTCCGCTCTGAGCAATCATGTGCAGTGCCAATATGGG |
| | AAA-3′ |
| | (SEQ ID NO: 55) |

The invention provides polynucleotide therapy useful for increasing the expression of miR-96, miR-182, or miR-183 microRNA, or any combination thereof for the treatment of ophthalmological and ear diseases. Expression vectors encoding a desired sequence (e.g. encoding a microRNA) can be delivered to cells of a subject having an ophthalmological or ear disease. The nucleic acid molecules must be delivered to the cells of a subject in a form in which they can be taken up and are advantageously expressed so that therapeutically effective levels can be achieved.

Methods for delivery of the polynucleotides to the cell according to the invention include using a delivery system such as liposomes, polymers, microspheres, gene therapy vectors, and naked DNA vectors.

Transducing viral (e.g., retroviral, adenoviral, lentiviral and adeno-associated viral) vectors can be used for somatic cell gene therapy, especially because of their high efficiency of infection and stable integration and expression. For example, a polynucleotide encoding a nucleic acid molecule can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Ban Virus. Retroviral vectors are particularly well developed and have been used in clinical settings (U.S. Pat. No. 5,399,346). Viral vectors are preferably replication incompetent in the cells to which they are delivered for therapeutic applications. However, replication competent viral vectors may also be used.

Preferred viral vectors for use in the invention include AAV vectors, e.g. AAV serotypes 1, 2, 3, 4, 5, 6, 7, 8, and/or 9, including chimeric AAV vectors. The availability of multiple AAV serotypes allows efficient targeting of many tissues of interest (Gao et al., 2002; McCarty, 2008; US Patent Publications 2008075737, 20080050343, 20070036760, 20050014262, 20040052764, 20030228282, 20030013189, 20030032613, and 20020019050 each incorporated herein by reference). In preferred embodiments, the invention includes the use of self-complementary (sc) AAV vectors which are described, for example, in US Patent Publications 20070110724 and 20040029106, and U.S. Pat. Nos. 7,465,583 and 7,186,699 (all of which are incorporated herein by reference). Exemplary methods for preparing AAVs for expressing microRNA are described in Knabel et al., PLoS One, 10(4):e0124411, 2015 and Xie et al., Semin Liver Dis, 35(1): 81-88, 2015 (both of which are incorporated herein by reference).

Non-viral approaches can also be employed for the introduction of a therapeutic nucleic acid molecule to a cell of a patient having an ophthalmological or ear disease. For example, an expression vector that encodes a miR-96, miR-182 and/or miR-183 microRNA can be introduced into a cell by administering the nucleic acid in the presence of lipofection, calcium phosphate co-precipitation, electroporation, microinjection, DEAE-dextran, transfection employing polyamine transfection reagents, cell sonication, gene bombardment using high velocity microprojectiles, and receptor-mediated transfection.

Nucleic acid molecule expression for use in polynucleotide therapy methods can be directed from any suitable promoter (e.g., the human cytomegalovirus (CMV), simian virus 40 (SV40), metallothionein, Ula1 snRNA. Ulb2 snRNA, histone H2, and histone H3 promoters), and regulated by any appropriate mammalian regulatory element. The expression may be directed using a tissue-specific or ubiquitously expressed promoter. Tissue specific promoters useful for the treatment of ophthalmological diseases include, but are not limited to, rhodopsin promoter, calcium binding protein 5 (CABP5) promoter, and cellular retinaldehyde binding protein (CRALBP) promoter. If desired, enhancers known to preferentially direct gene expression in specific cell types can be used to direct the expression of a nucleic acid. The enhancers used can include, without limitation, those that are characterized as tissue- or cell-specific enhancers.

In one embodiment, an expression vector for expressing an agonist of miR-96, miR-182, or miR-183 comprises a promoter and a terminator operably linked to a polynucleotide sequence encoding an agonist, wherein the expressed agonist comprises a first strand comprising a mature sequence of miR-96 (SEQ ID NOs: 1, 2, or 3), miR-182 (SEQ ID NOs: 4, 5, or 6), or miR-183 (SEQ ID NOs: 7, 8, or 9) and a second strand that is substantially complementary to the first strand. In another embodiment, an expression vector for expressing an agonist of miR-96, miR-182, or miR-183 comprises a promoter and a terminator operably linked to a polynucleotide sequence encoding a pre-miRNA sequence, wherein the expressed agonist comprises a polynucleotide sequence in the form of a hairpin comprising a mature miRNA sequence. In yet another embodiment, an expression vector for expressing an agonist of miR-96, miR-182, or miR-183 comprises a first promoter and a first terminator operably linked to a first polynucleotide sequence encoding an antisense strand of miR-96, miR-182, or miR-183 mimetic compound and a second promoter and a second terminator operably linked to a second polynucleotide sequence encoding a sense strand of miR-96, miR-182, or miR-183 mimetic compound. The phrase ‘operably linked’ or ‘under transcriptional control’ as used herein means that the promoter and the terminator are in the correct location and orientation in relation to a polynucleotide to control the initiation and termination of transcription by RNA polymerase and expression of the polynucleotide.

As used herein, a “promoter” refers to a DNA sequence recognized by the synthetic machinery of the cell, or introduced synthetic machinery, required to initiate the specific transcription of a gene. Suitable promoters include, but are not limited to RNA pol I, pol II, pol III, and viral promoters (e.g. human cytomegalovirus (CMV) immediate early gene promoter, the SV40 early promoter, and the Rous sarcoma virus long terminal repeat). In one embodiment, the promoter is a tissue-specific promoter. Of particular interest are retinal cell specific promoters, and more particularly, rods and cones specific promoters. These include the rhodopsin promoter, cone opsin promoter, calcium binding protein 5 (CABP5) promoter, cellular retinaldehyde binding protein (CRALBP) promoter, interphotoreceptor retinoid-binding protein (IRBP) promoter, arrestin promoter, and rhodopsin kinase promoter. As used herein, a “terminator” refers to a DNA sequence recognized by the synthetic machinery of the cell, or introduced synthetic machinery, that is required to terminate the specific transcription of a gene. In one embodiment, a sequence comprising a polyadenylation signal/unit acts as a transcription terminator. The use of other transcription terminators is also contemplated.

In certain embodiments, the promoter operably linked to a polynucleotide encoding an agonist of miR-96, miR-182, or miR-183 can be an inducible promoter. Inducible promoters are known in the art and include, but are not limited to, tetracycline promoter, metallothionein HIA promoter, heat shock promoter, steroid/thyroid hormone/retinoic acid response elements, the adenovirus late promoter, and the inducible mouse mammary tumor virus LTR.

### Treatment Methods

In various embodiments, the present invention provides methods of treating or preventing ophthalmological conditions in a subject in need thereof comprising administering to the subject a therapeutically effective amount of at least one agonist of miR-96, miR-182, and/or miR-183. In some embodiments, methods of treating or preventing ophthalmological conditions in a subject in need thereof comprise administering to the subject therapeutically effective amounts of at least two agonists, for example, agonists of miR-96 and miR-182, agonists of miR-96 and miR-183, or agonists of miR-182 and miR-183. In other embodiments, methods of treating or preventing ophthalmological conditions in a subject in need thereof comprise administering to the subject therapeutically effective amounts of all three agonists, i.e., agonists of miR-96, miR-182, and miR-183. In some embodiments, the agonist of miR-96, miR-182, or miR-183 is a double-stranded oligonucleotide comprising a first strand containing a sequence of mature miR-96, miR-182, or miR-183 and a second strand comprising a sequence that is substantially complementary to the first strand, wherein at least one of the strands comprises one or more modified nucleotides. Any of the microRNA mimetic compounds described herein can be used in the methods of treating or preventing an ophthalmological condition in a subject in need thereof. In some embodiments, the agonist of miR-96, miR-182, or miR-183 is an expression vector comprising a polynucleotide sequence encoding a sufficient portion of the miR-96, miR-182, or miR-183 native coding sequence to produce a mature miR-96, miR-182, or miR-183. In one embodiment, the expression vector is a recombinant AAV vector.

A “therapeutically effective amount or dose” is an amount sufficient to effect a beneficial or desired clinical result. For example, a therapeutically effective amount of microRNA mimetic compounds of miR-96, miR-182, and/or miR-183 includes an amount that is sufficient to maintain or improve visual acuity or an amount that is sufficient to reduce or prevent loss of vision or an amount that is sufficient to reduce or prevent photoreceptor cell death. In another embodiment, a therapeutically effective amount is an amount sufficient to increase expression of one or more phototransduction genes in the photoreceptor cells of the subject.

Ophthalmological conditions that may be treated by administering one or more agonists of miR-96, miR-182, and miR-183, include any condition caused by damage and/or death of photoreceptor cells. The term “photoreceptor cell” includes rods, cones, ganglion cells as well as other cells (e.g. retinal cells) present in the eye. In certain embodiments, ophthalmological conditions caused by damage or death of photoreceptor cells include retinal detachment, macular degeneration, Stargardt disease, retinal degeneration, retinitis pigmentosa, night blindness, retinal toxicity, uveal melanoma, sympathetic ophthalmia and retinopathies. In certain embodiments, the ophthalmological condition that may be treated according to the invention is retinal degeneration. In one embodiment, the ophthalmological condition that may be treated according to the invention is retinitis pigmentosa. In another embodiment, a subject in need of treatment with an agonist of miR-96, miR-182, and/or miR-183 may have signs of night blindness.

In various embodiments, administration of at least one agonist of miR-96, miR-182, or miR-183 to the subject prevents or slows the development and/or progression of one or more ophthalmological conditions and results in the improvement of one or more symptoms associated with these conditions. For instance, in one embodiment, administration of at least one agonist of miR-96, miR-182, or miR-183 results in the improvement of visual acuity. In another embodiment, administration of at least one agonist of miR-96, miR-182, or miR-183 reduces the signs of night blindness. In yet another embodiment, administration of at least two agonists, for example, agonists of miR-96 and miR-182, agonists of miR-96 and miR-183, or agonists of miR-182 and miR-183, results in the improvement of visual acuity. In still another embodiment, administration of all three agonists, i.e., agonists of miR-96, miR-182, and miR-183, results in the improvement of visual acuity.

In certain embodiments, the present invention provides methods for ameliorating or restoring visual acuity in a subject in need thereof comprising administering to the subject at least one agonist of miR-96, miR-182, and/or miR-183. According to the invention, systemic, local or topical administration of at least one agonist of miR-96, miR-182, or miR-183 to a subject in need thereof results in the increased activity of miR-96, miR-182, and/or miR-183 in various eye cells, such as rods, cones, Müller cells, horizontal cells, bipolar cells, amacrine cells, and/or ganglion cells of the subject.

In one embodiment, administration of at least one agonist of miR-96, miR-182, and/or miR-183 maintains or improves the function of photoreceptor cells such as rods and cones and/or other retinal cells, maintains or improves visual acuity, reduces or prevents the death of photoreceptor cells, and/or reduces or prevents vision loss. In certain embodiments, administration of at least one agonist of miR-96, miR-182, and/or miR-183 maintains or increases the expression of one or more phototransduction genes in the photoreceptor cells of the subject. The one or more phototransduction genes that may be upregulated upon administration of at least one agonist of miR-96, miR-182, and/or miR-183 include Recoverin (Revrn), NRL, Arrestin (Sag). Rhodopsin (Rho), Transducin (Gnat2), and Phosducin (PDC).

In one embodiment, vision loss, visual acuity, and/or retinal degeneration in a subject is measured using tests such as optokinetic tracking (OKT), electroretinography (ERG), mean spatial frequency threshold (SFT), and measurement of the thickness of the Inner and Outer Nuclear Cell layers of the retina. In another embodiment, a subject's visual acuity is determined using a protocol such as the Early Treatment for Diabetic Retinopathy Study (“ETDRS”) or the Age-Related Eye Disease Study (“AREDS”) protocol. In some embodiments, visual acuity is measured using a modified ETDRS and/or AREDS protocol, such as the measurement of visual acuity described in Ferris et al., Am J Ophthalmol 94:91-96, 1982. In one embodiment, a subject's visual acuity is determined by one or more of the following procedures: (1) measurement of best-corrected visual acuity (BCVA) with required manifest refraction; (2) measurement of corrected visual acuity with conditional manifest refraction; or (3) measurement of corrected visual acuity without manifest refraction. In various aspects of the invention, administration of at least one agonist of miR-96, miR-182, or miR-183 to a subject in need thereof results in improved scores on one or more of these eye tests. In some embodiments, administration of at least two agonists, for example, agonists of miR-96 and miR-182, agonists of miR-96 and miR-183, or agonists of miR-182 and miR-183, to a subject results in improved scores on one or more of the eye tests. In some other embodiments, administration of all three agonists, i.e., agonists of miR-96, miR-182 and miR-183, to a subject results in improved scores on one or more of the eye tests. For instance, in one embodiment, a subject upon administration with at least one, at least two, or all three agonists of miR-96, miR-182, and miR-183 has a greater than 3-line, 4-line or 5-line gain in visual acuity in a standardized chart of visual testing, e.g., the ETDRS chart. In another embodiment, administration of at least one, at least two, or all three agonists of miR-96, miR-182, and miR-183 results in the subject's ability to read one or more additional, in some embodiments three or more additional, and in some embodiments 15 or more additional, letters of a standardized chart of vision testing, e.g., the Early Treatment for Diabetic Retinopathy Study Chart (“ETDRS chart”).

In some embodiments, the present invention provides methods of treating or preventing diseases or disorders of other sensory organs in a subject in need thereof comprising administering to the subject a therapeutically effective amount of at least one agonist of miR-96, miR-182, and/or miR-183. For example, in one embodiment, the present invention provides methods of treating or preventing diseases or disorders of ear such as hearing loss, tinnitus. Meniere's disease, ear infections, or damage caused to the ear by these conditions. In some embodiments, methods of treating or preventing ear disorders in a subject in need thereof comprise administering to the subject therapeutically effective amounts of at least two agonists, for example, agonists of miR-96 and miR-182, agonists of miR-96 and miR-183, or agonists of miR-182 and miR-183. In other embodiments, methods of treating or preventing ear disorders in a subject in need thereof comprise administering to the subject therapeutically effective amounts of all three agonists, i.e., agonists of miR-96, miR-182, and miR-183. In some embodiments, the agonist of miR-96, miR-182, or miR-183 is a double-stranded oligonucleotide comprising a first strand containing a sequence of mature miR-96, miR-182, or miR-183 and a second strand comprising a sequence that is substantially complementary to the first strand, wherein at least one of the strands comprises one or more modified nucleotides. Any of the microRNA mimetic compounds described herein can be used in the methods of treating or preventing an ear disorders in a subject in need thereof. In some embodiments, the agonist of miR-96, miR-182, or miR-183 is an expression vector comprising a polynucleotide sequence encoding a sufficient portion of the miR-96, miR-182, or miR-183 native coding sequence to produce a mature miR-96, miR-182, or miR-183. In one embodiment, the expression vector is a recombinant AAV vector.

In various embodiments, administration of at least one agonist of miR-96, miR-182, or miR-183 to the subject prevents or slows the development and/or progression of one or more ear disorders and results in the improvement of one or more symptoms associated with these conditions. For instance, in one embodiment, administration of at least one agonist of miR-96, miR-182, or miR-183 results in the improvement of hearing ability. In another embodiment, administration of at least one agonist of miR-96, miR-182, or miR-183 improves the function of sensory cells of the ear. In yet another embodiment, administration of at least two agonists, for example, agonists of miR-96 and miR-182, agonists of miR-96 and miR-183, or agonists of miR-182 and miR-183, results in the improvement of hearing ability and/or the function of ear cells. In still another embodiment, administration of all three agonists, i.e., agonists of miR-96, miR-182, and miR-183, results in the improvement of hearing ability and/or the function of ear cells.

As used herein, the term “subject” or “patient” refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In other embodiments, the subject is a human.

### Pharmaceutical Compositions

The present invention also provides pharmaceutical compositions comprising a therapeutically effective amount of one or more agonists of miR-96, miR-182, and/or miR-183 according to the invention and a pharmaceutically acceptable carrier or excipient. In one embodiment, the present invention provides pharmaceutical compositions comprising a therapeutically effective amount of one or more synthetic microRNA mimetic compounds of miR-96, miR-182, and/or miR-183 according to the invention or a pharmaceutically acceptable salt thereof, and a pharmaceutically acceptable carrier or excipient. In other embodiments, the invention provides pharmaceutical compositions comprising one or more expression vectors encoding miR-96, miR-182, and/or miR-183 and a pharmaceutically acceptable carrier or excipient, wherein the amount of the expression vectors provides therapeutically effective amount of miR-96, miR-182, and/or miR-183.

The term “pharmaceutically acceptable salt” refers to a salt prepared by combining a compound, such as the disclosed miRNA mimetic compounds, with an acid whose anion, or a base whose cation, is generally considered suitable for human consumption. Suitable pharmaceutically acceptable acid addition salts of the disclosed compounds include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids.

Suitable organic acids generally include, for example, aliphatic, cycloaliphatic, aromatic, araliphatic, heterocyclylic, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, malcate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, O-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Furthermore, where the disclosed compounds carry an acidic moiety, suitable pharmaceutically acceptable salts thereof can include alkali metal salts, e.g., sodium or potassium salts; alkaline earth metal salts, e.g., calcium or magnesium salts; and salts formed with suitable organic ligands, e.g., quaternary ammonium salts. In some forms, base salts are formed from bases which form non-toxic salts, including aluminum, arginine, benzathine, choline, diethylamine, diolamine, glycine, lysine, meglumine, olamine, tromethamine and zinc salts.

Organic salts can be made from secondary, tertiary or quaternary amine salts, such as tromethamine, diethylamine. N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (C1-C6) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others. In some forms, hemisalts of acids and bases can also be formed, for example, hemisulphate and hemicalcium salts. In certain embodiments, a pharmaceutically acceptable salt of the present mimetic compounds include a sodium salt.

In one embodiment, the pharmaceutical composition comprises a therapeutically effective amount of a miR-96 mimetic compound and a pharmaceutically acceptable carrier or excipient, wherein the first strand of the mimetic compound comprises a mature miR-96 sequence and the second strand is substantially complementary to the first strand. In another embodiment, the pharmaceutical composition comprises a therapeutically effective amount of a miR-182 mimetic compound and a pharmaceutically acceptable carrier or excipient, wherein the first strand of the mimetic compound comprises a mature miR-182 sequence and the second strand is substantially complementary to the first strand. In yet another embodiment, the pharmaceutical composition comprises a therapeutically effective amount of a miR-183 mimetic compound and a pharmaceutically acceptable carrier or excipient, wherein the first strand of the mimetic compound comprises a mature miR-183 sequence and the second strand is substantially complementary to the first strand.

In some embodiments, the pharmaceutical composition comprises a therapeutically effective amount of at least two microRNA mimetic compounds of the invention and a pharmaceutically acceptable carrier or excipient, wherein the first strand of the first microRNA mimetic compound comprises a mature miR-96 sequence and the first strand of the second microRNA mimetic compound comprises a mature miR-182 sequence. In some other embodiments, the pharmaceutical composition comprises a therapeutically effective amount of at least two microRNA mimetic compounds of the invention and a pharmaceutically acceptable carrier or excipient, wherein the first strand of the first microRNA mimetic compound comprises a mature miR-96 sequence and the first strand of the second microRNA mimetic compound comprises a mature miR-183 sequence. In still some other embodiments, the pharmaceutical composition comprises a therapeutically effective amount of at least two microRNA mimetic compounds of the invention and a pharmaceutically acceptable carrier or excipient, wherein the first strand of the first microRNA mimetic compound comprises a mature miR-182 sequence and the first strand of the second microRNA mimetic compound comprises a mature miR-183 sequence. In yet some other embodiments, the invention provides pharmaceutical compositions comprising a therapeutically effective amount of three microRNA mimetic compounds of the invention and a pharmaceutically acceptable carrier or excipient, wherein the first strand of the first microRNA mimetic compound comprises a mature miR-96 sequence, the first strand of the second microRNA mimetic compound comprises a mature miR-182 sequence, and the first strand of the third microRNA mimetic compound comprises a mature miR-183 sequence.

Preferably, in the pharmaceutical compositions comprising at least two microRNA agonists according to the invention, the first and the second agonists or the first, second and the third agonists are present in equimolar concentrations. Other mixing ratios such as about 1:2, 1:3, 1:4, 1:5, 1:2:1, 1:3:1, 1:4:1, 1:2:3, 1:2:4 are also envisioned for preparing pharmaceutical compositions comprising at least two of the miR-96, miR-182, and miR-183 agonists.

In some embodiments, one or more microRNA agonists of the invention may be administered concurrently but in separate compositions, with concurrently referring to mimetic compounds given within short period, for instance, about 30 minutes of each other. In some other embodiments, miR-96, miR-182, and/or miR-183 agonists may be administered in separate compositions at different times.

The invention also encompasses embodiments where additional therapeutic agents may be administered along with miR-96, miR-182, and/or miR-183 agonists. The additional therapeutic agents may be administered concurrently but in separate formulations or sequentially. In other embodiments, additional therapeutic agents may be administered at different times prior to after administration of miR-96, miR-182, and/or miR-183 agonists. Prior administration includes, for instance, administration of the first agent within the range of about one week to up to 30 minutes prior to administration of the second agent. Prior administration may also include, for instance, administration of the first agent within the range of about 2 weeks to up to 30 minutes prior to administration of the second agent. After or later administration includes, for instance, administration of the second agent within the range of about one week to up to 30 minutes after administration of the first agent. After or later administration may also include, for instance, administration of the second agent within the range of about 2 weeks to up to 30 minutes after administration of the first agent. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

In some embodiments, pharmaceutical compositions comprising the miR-96, miR-182, and/or miR-183 agonists can be formulated for topical ophthalmic application, for example, in the form of solutions, ointments, creams, lotions, eye ointments, eye drops or eye gels. In some other embodiments, pharmaceutical compositions comprising the miR-96, miR-182, and/or miR-183 agonists can be formulated for local ocular administration via injection. The routes for administering the miR-96, miR-182, and/or miR-183 agonists via injection include intravitreal, peri-ocular, intracameral, subconjunctival, or transcleral administration.

In various embodiments, pharmaceutical compositions used for topical or local ocular administration can contain appropriate ophthalmological additives including, for example, buffers, isotonizing agents, preservatives, solubilizers (stabilizers), pH adjusting agents, thickeners and chelating agents, solvents to assist drug penetration, and emollients in ointments and creams. The buffers may be selected from but not limited by the group comprising a phosphate buffer, a borate buffer, a citrate buffer, a tartrate buffer, an acetate buffer (for example, sodium acetate) and an amino acid. The isotonizing agents may be selected from but not limited by the group comprising sugars such as sorbitol, glucose and mannitol, polyhydric alcohols such as glycerin, polyethylene glycol and polypropylene glycol, and salts such as sodium chloride. The preservatives may be selected from but not limited by the group comprising benzalkonium chloride, benzethonium chloride, alkyl paraoxybenzoates such as methyl paraoxybenzoate and ethyl paraoxybenzoate, benzyl alcohol, phenethyl alcohol, sorbic acid and salts thereof, thimerosal and chlorobutanol. The solubilizers (stabilizers) may be selected from but not limited by the group comprising cyclodextrin and derivatives thereof, water-soluble polymers such as poly(vinylpyrrolidone), and surfactants such as polysorbate 80 (trade name: Tween 80). The pH adjusting agents may be selected from but not limited by the group comprising hydrochloric acid, acetic acid, phosphoric acid, sodium hydroxide, potassium hydroxide and ammonium hydroxide. The thickeners may be selected from but not limited by the group comprising hydroxyethylcellulose, hydroxypropylcellulose, methylcellulose, hydroxypropylmethylcellulose and carboxymethylcellulose and salts thereof. The chelating agents may be selected from but not limited by the group comprising sodium edetate, sodium citrate and sodium condensed phosphate. Such topical formulations can further contain compatible ophthalmic carriers, for example cream or ointment bases, olive oil, arachis oil, castor oil, polyoxyethylated castor oil, mineral oil, petroleum jelly, dimethyl sulphoxide, an alcohol (ethanol or oleyl alcohol), liposome, silicone fluid and mixtures thereof as taught by U.S. Pat. No. 6,254,860.

Alternatively, the miR-96, miR-182, and miR-183 agonists may be applied to the eye via liposomes. In certain embodiments, liposomes used for delivery are amphoteric liposomes such SMARTICLES@ (Marina Biotech, Inc.) which are described in detail in U.S. Pre-grant Publication No. 20110076322. The surface charge on the SMARTICLES@ is fully reversible which make them particularly suitable for the delivery of nucleic acids. SMARTICLES® can be delivered via injection, remain stable, and aggregate free and cross cell membranes to deliver the nucleic acids.

Further, the agonists may be infused into the tear film via a pump-catheter system. Another embodiment of the present invention involves the mimetic compounds contained within a continuous or selective-release device, for example, membranes such as, but not limited to, those employed in the pilocarpine (OCUSERT™) System (Alza Corp., Palo Alto, Calif.). As an additional embodiment, the mimetic compounds can be contained within, carried by, or attached to contact lenses which are placed on the eye. Another embodiment of the present invention involves the mimetic compounds contained within a swab or sponge which can be applied to the ocular surface. Yet another embodiment of the present invention involves the mimetic compounds contained within a liquid spray which can be applied to the ocular surface.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

All patent and non-patent documents referenced throughout this disclosure are incorporated by reference herein in their entirety for all purposes.

### EXAMPLES

### Example 1: Uptake and Clearance of miRNA Mimics in Retina

Naked (not conjugated to cholesterol), miRNA mimics for mouse miR-96, miR-182, and miR-183 were resuspended in saline. The three duplexes were pooled at 3.3 μg/μl of each duplex to obtain 10 μg/μl of pooled duplexes. Naked siRNA targeting mouse rhodopsin (NM_145383) was used as a positive control and was suspended in saline at a concentration of 10 μg/μl. To determine the uptake and clearance of miRNA mimics in retina, wild-type C57Bl/6 mice were injected intravitreally with 1 μl (10 μg) of pooled miRNA duplexes or rhodopsin siRNA per eye. Retina was isolated after each time point and the retinal distribution and clearance was measured by isolating RNA and measuring miRNA levels by sandwich ELISA. For the distribution and clearance of siRNA, RNA was isolated and target repression was measured by qRT-PCR.

Study Design:

### 2 Study Arms:

1) Pool of miR-96/182/183 mimics, 10 μg total (n=14)
2) siRNA targeting rhodopsin (n=14)

### 7 Time Points: (2 Mice/Study Arm/Timepoint)

1) Untreated (baseline)
2) 4 h
3) 8 h
4) 24 h
5) 48 h
6) 72 h
7) 168 h (day 7)

miRNA Mimic/siRNA Quantitation Assay to Assess Oligonucleotide Biodistribution

A sandwich hybridization assay was used to quantify miR-183, miR-96, miR-182, or rho siRNA in tissue samples as previously described by Efler, et al (“Quantitation of oligodeoxynucleotides in human plasma with a novel hybridization assay offers greatly enhanced sensitivity over capillary electrophoresis,” Oligonucleotides 15(2), 119-131 (2005)). Briefly, probes for the hybridization assay were synthesized with 2′-O-methyl modified nucleotides and were tagged as 5′ bTEG-sup-3′ (capture probe) and 5′-6FAM-sup-3′ (detection probe). Detection was accomplished using anti-fluorescence-peroxidase, Fab fragments (Roche), and TMB Peroxidase Substrate (KPL). Standard curves were generated with nonlinear logistic regression analysis with 4 parameters (4-PL). The working concentration range of the assay was 1 to 536 ng/mL. Tissue samples were prepared at 100 mg/mL by homogenizing in 3 mol/L GITC buffer (3 mol/L guanidineisothiocyanate, 0.5 mol/L NaCl, 0.1 mol/L Tris, pH 7.5, and 10 mmol/L EDTA) two times for 30 seconds with an MP FastPrep-24 at a speed setting of 6.0. Tissue homogenates were diluted in 1 mol/L GITC Buffer (1 mol/L guanidine isothiocyanate, 0.5 mol/L NaCl, 0.1 mol/L Tris, pH 7.5, and 10 mmol/L EDTA) for testing.

Quantitative Real-Time Polymerase Chain Reaction Analysis of Mouse Rhodopsin

For in vivo real-time polymerase chain reaction (RT-PCR) analysis, RNA was extracted from retina tissue with Trizol (Invitrogen); then, 100 ng total RNA from each sample was used to generate cDNA with MultiScribe reverse transcriptase (Life Technologies) according to the manufacturer's specifications. Gene expression was measured with Life Technologies Taqman gene expression assays. Gene expression was normalized to a housekeeping gene such as GAPDH and calculated as relative expression compared to the average of the control group.

For animals treated with miRNA mimic pool, 2 retinas from each animal were pooled for biodistribution analysis. For animals treated with Rho siRNA, one retina per animal was used for biodistribution, and the other retina was used for RNA isolation to quantitate target knockdown.

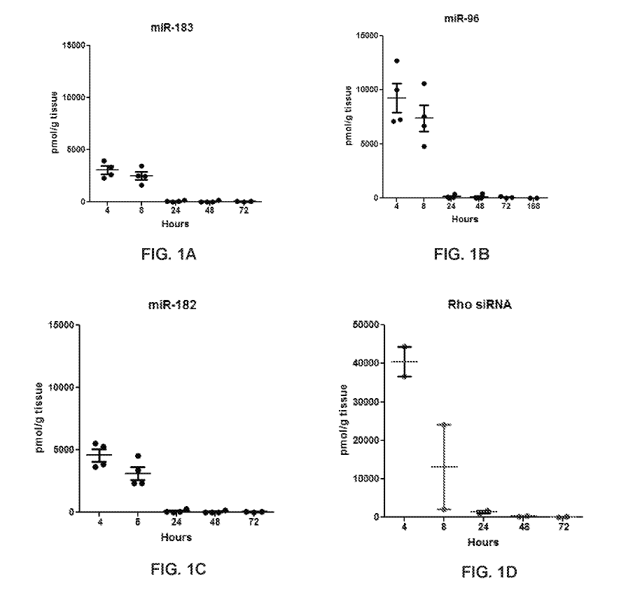

All 3 miRNA mimics and the Rho siRNA distributed to the retina after a single intravitreal injection. The amount of each miRNA mimic is approximately one-third the amount of siRNA detected in retina, consistent with the fact that the miRNA mimic pool contained 3.3 μg of each miRNA mimic (10 μg total oligo), while the siRNA was dosed at a total concentration of 10 μg. All three miRNA mimics were detected at 4 h and 8 h post-dose and were cleared rapidly after 8 h (FIG. 1). Rho siRNA was detected at 4 h and 8 h post-injection and was cleared by 24 h.

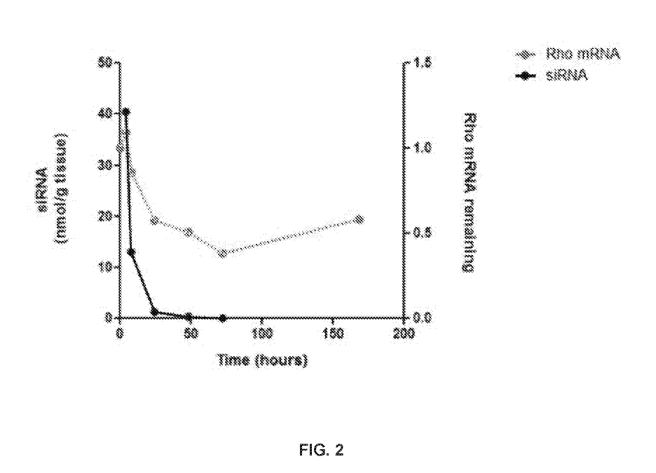

The Rho (rhodopsin) siRNA produced 60% silencing of the target gene at 72 h post-injection, and silencing remained at ˜50% at 7 days post-injection (FIG. 2). Rhodopsin is expressed in photoreceptors in the retina. The current data therefore demonstrate that siRNA duplexes distribute functionally to photoreceptors. Silencing is retained for ˜1 week post-injection, even though the siRNA itself is not detectable in the retina after 24 h.

### Example 2: Administration of microRNA Mimics (miR-96, miR-182, and miR-183) in Rat Retinal Cell System

For this study, cholesterol-conjugated, mimics for miR-96, miR-182, and miR-183, and rat rhodopsin siRNA were used.

TABLE 3
miR-96/182/183 mimics used in the study:

| SEQ | |
|---|---|
| ID NO. | Modified Sequence |
| 13 | 5′-mA.mG.mC.rA.rA.rA.rA.mU.mU.rG.rA.rG. |
| | mC.mU.rA.rG.mU.rG.mC.rG.rA.rA.rA. |
| | chol6-3′ |
| 18 | 5′-mA.mG.mU.rG.mU.rG.rA.rG.rA.mU.mC.rA. |
| | rA.mC.mC.rA.mU.mU.rG.mC.rG.rA.rA.rA. |
| | chol6-3′ |
| 23 | 5′-mA.mG.mU.rG.rA.rA.rA.mU.mC.rA.rA.mC. |
| | mC.rA.rG.mU.rG.mC.rG.rA.mU.rA.chol6-3′ |
| 20 | 5′-rU.rA.fU.rG.rG.fC.rA.fC.fU.rG.rG.fU. |
| | rA.rG.rA.rA.fU.fU.fC.rA.fC.fUs.rUs.rU- |
| | sup-3′ |
| 10 | 5′-rU.rU.fU.rG.rG.fC.rA.fC.fU.rA.rG.fC. |
| | rA.fC.rA.fU.fU.fU.fU.fU.rG.fC.fUs.rUs. |
| | rU-sup-3′ |
| 15 | 5′-rU.rU.fU.rG.rG.fC.rA.rA.fU.rG.rG.fU. |
| | rA.rG.rA.rA.fC.fU.fC.rA.fC.rA.fC.fUs. |
| | rUs.rU-sup-3′ |

Cholesterol-conjugated mimics of each of the 3 microRNAs were administered to rat retinal cells purchased from Lonza (R-RET-508). Lonza's rat retinal cell system isolated from neonatal (P3 or P4) Sprague-Dawley rats comprises 7 cell types normally found in the retina (rods, cones, Miller cells, horizontal cells, bipolar cells, amacrine cells, and ganglion cells). Although this is a mixed cell population, data on the proportions of cell types in the rat retina indicate that rods are the predominant cell type, and rods are also the cell type in which the miR-183 cluster is normally most highly expressed. Studying the effect of miRNA mimics in the context of other retinal neuronal cell types could provide more relevant, context-dependent, biology compared to a single cell type in isolation. Additionally, this mixed cell system could provide a more direct comparison for the gene expression changes identified in subsequent in vivo studies. At P3/P4 neonatal stage in the rats, the microRNAs in the miR-183/96/182 cluster are relatively lowly expressed (˜1% of levels in the adult retina) thereby providing low background and a more robust signature for exogenously added mimic.

Rat retinal cells (R-Ret cells) in culture were passively transfected with cholesterol-conjugated microRNA mimics to determine various parameters: toxicity of miRNA mimics, down-regulation of Rho mRNA using Rho siRNA, optimal plating density of R-Ret cells, maximum duration for culturing R-Ret cells, and expression profiling of genes involved in the phototransduction pathway using real-tile PCR.

Study Design:
A. Determine the highest dose of cholesterol conjugated miRNA mimic tolerated by R-Ret cells with 10 μM being the highest dose administered.
B. Assess functional uptake by measuring Rho knockdown using cholesterol-conjugated siRNA pool
C. Determine the optimal plating density of R-Ret cells that yields at least 800 ng of total RNA (the minimum required for microarray profiling on the Affymetrix platform)
D. Determine maximal amount of time cells can be cultured
E. Perform real time PCR on a selection of genes that are functionally involved in the phototransduction pathway

End Points:
A. Toxicity/viability assay and visual assessment
B. qPCR of Rho
C. UV/Visible RNA quantification
D. miRNA qPCR
E. qPCR of Rho. Sag, Arr3. Rcvrn, Nrl, Pdc, Gnat1, Gnat2, Opn1mw

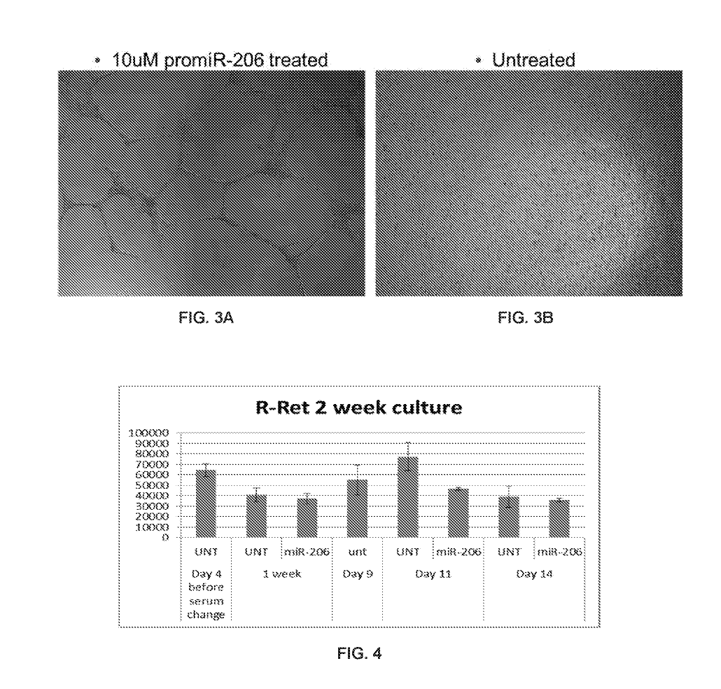

R-Ret cells were passively transfected with cholesterol-conjugated miR-206 mimic and the cells were observed 72 hours post-transfection. 10 μM dose of cholesterol-conjugated miRNA mimic appeared to cause differentiation of the cells, 72 hours post-transfection and 1 week after start of the culture (FIG. 3). Toxicity of miRNA mimic on R-Ret cells was measured using an adenylate kinase assay. Toxicity was found to decrease over time in serum-free media (FIG. 4) and the treatment of cells with 10 μM miRNA mimic was not found to be toxic.

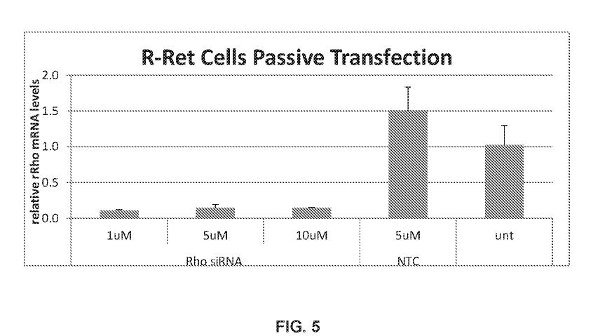

R-Ret cells were passively transfected with various concentrations of cholesterol-conjugated Rho siRNA or non-targeting control (NTC) siRNA. RNA was isolated and a real-time PCR analysis of Rho mRNA was performed. FIG. 5 shows that 1, 5, and 10 μM of cholesterol-conjugated Rho siRNA pool produced comparable Rho knockdown.

RNA was isolated from R-Ret cells cultured for one week and yield was determined using a UV/visible spectrophotometer (Table 4).

TABLE 4
Using miRNesay columns/qiazol

| | Day after | | Plate | elution | ng/uL | ng/uL |
|---|---|---|---|---|---|---|
| Cell | plating | Density | well | volume | 1st elute | 2nd elute |
| R-Ret | 1 week | 140K | 12 | 30 | 60.7 | 39.4 |

To determine the maximum amount of time cells can be cultured, R-Ret cells were plated on poly-L-lysine. Cells survived for at least two weeks in culture where the cells were cultured in 5% serum media for first four days and then the media was changed to serum free media.

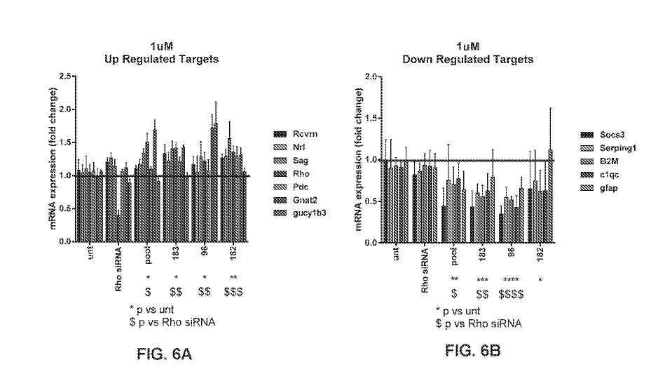

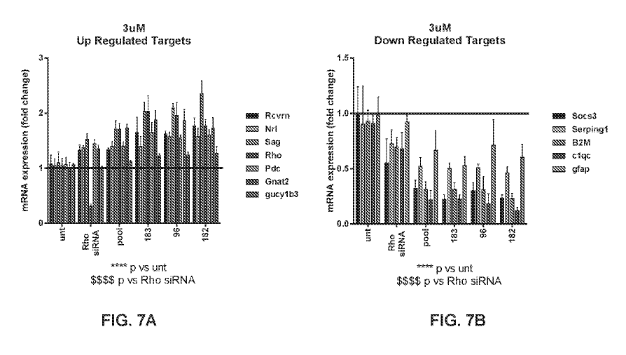

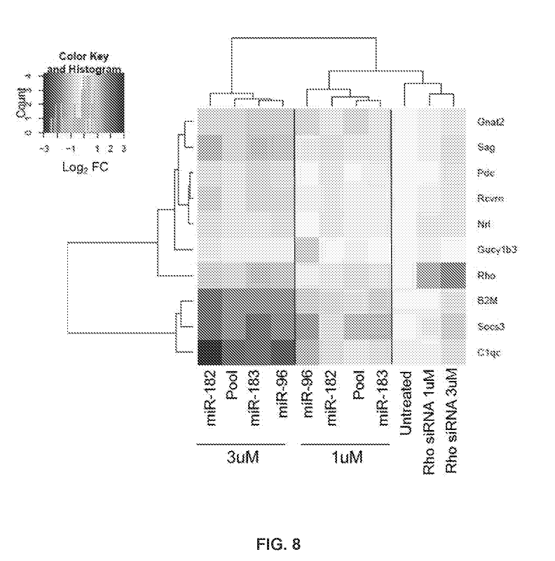

R-Ret cells were transfected with various concentrations of pooled or individual miR-183, miR-96, and miR-182 mimics and Rho siRNA. RNA was isolated 72 hours post-transfection and real time PCR analysis was performed to determine the mRNA expression profile of genes involved in the phototransduction pathway. FIGS. 6 and 7 show relative expression levels of genes that are expressed within the range of the PCR assay. P-values of difference were calculated by two-way ANOVA with Newman-Keuls test for multiple comparisons compared to the untreated group. FIG. 8 shows the heat map of the log 2-transformed average fold change values of the treatments shown in FIG. 7.

TABLE 5
Genes examined by real time PCR

| | | | Expression level |
|---|---|---|---|
| Gene name | Gene symbol | Cell type expression | (real time) |
| Rhodopsin | Rho | Rod-specific | Good |
| Arrestin | Sag | Rod-specific | Good |
| Transducin | Gnat1 | Rod-specific | Low |
| Phosducin | PDC | Rod-specific | Good |
| NRL | NRL | Rod-specific | Good |
| Transducin | Gnat2 | Cone-specific | Good |
| M-cone opsin | Opn1mw | Cone-specific | Not detectable |
| Arrestin | Arr3 | Cone-specific | Low |
| Recoverin | Revrn | Rod and cone | Good |

Table 6 shows a comparison of the expression data of selected genes in the miR-183 cluster knockout mouse with that of rat retinal cells treated with cholesterol-conjugated miR-183 cluster miRNA mimics.

TABLE 6

| | miR-183 cluster | Mimics of miR-183 | |
|---|---|---|---|
| Gene | Knock-out | cluster in vitro | Rho siRNA |
| Rcvm | — | ↑ | — |
| Nrl | ↓ | ↑ | — |
| Sag | — | ↑ | — |
| Rho | ↓ | ↑ | ↓ |
| Pdc | — | ↑ | — |
| Gnat2 | ↓ | ↑ | — |

### Example 3: Identification of Direct and Downstream Targets of microRNA Mimics (miR-96, miR-182, miR-183) in Retina

R-Ret cells in culture are passively transfected with cholesterol-conjugated microRNA mimics (miR-183, miR-96, miR-182) individually or pooled or with cholesterol-conjugated Rho siRNA. At various time points post-transfection, RNA is isolated and subjected to microarray profiling to identify significantly regulated transcripts.

### Example 4: Administration of microRNA Mimics Prevents Vision Loss and Retinal Degeneration

The effect of microRNA mimetic compounds (pool of microRNA mimics of miR-96 and miR-182, and a control duplex) on vision loss and retinal degeneration in a mouse model of retinitis pigmentosa was examined. Rho siRNA was used as a positive control.

Rd10/rd10 mice were dark reared to P31, at which point they were moved to a 12 hour light/dark cycle to induce retinal degeneration. At P31, the test agents (microRNA pool or Rho siRNA) were administered via bilateral intravitreal (IVT) injection at a concentration of 10 μg (Rho siRNA), or 2 μg and 10 μg (microRNA pool of miR-96 and miR-182, and a control duplex). Control groups included rd10/rd10 mice receiving bilateral administration of vehicle at P31, or animals receiving intraperitoneal (i.p.) administration of phenyl-N-tert-butylnitrone (PBN) daily from P29 to P35. For assessment of vision loss by optokinetic tracking (OKT) and electroretinography (ERG), an additional control group of untreated C57Bl/6J mice was included. Vision loss was assessed by both visual acuity measurements (P38 and P45) and ERG (P39 and P46). Visual acuity was assessed by determining the mean spatial frequency threshold (SFT) at which mice could distinguish visual stimuli presented in a virtual environment.

Administration of both 10 μg of pooled mimics (miR-96 and miR-182 mimics, and a control duplex) and PBN to rd10/rd10 mice resulted in a statistically significant retention of visual acuity as determined by OKT at P45.

Experimental Design

rd10/rd10 mice
P1-P30: Animals dark-reared from birth
P29-P35: Daily intraperitoneal administration of PBN (Arm 5)
P31: Animals transferred to housing in normal cyclic light (˜200 lux during daylight hours)
P31: Bilateral intravitreal dosing of vehicle or test agents (Arms 1-4)
P38: OKT analyses to quantify spatial frequency threshold
P45: OKT analyses to quantify spatial frequency threshold

Animals
Strain: rd10/rd10 mice
Sex: Male/Female
Age Range: Newborn pubs
Weight Range: n/a
Supplier: In-house breeding
Number of Study Animals: 48
Number of Spare Animals 0
Number of Sentinel Animals: 0

Test Compounds and Vehicle

TABLE 7

| | | Route of |
|---|---|---|
| Compound | Preparation & Storage | Administration |
| Pool of miR-96 mimic, | Store aliquots at −20° C. | Intravitreal |
| miR-182 mimic, and | until use. | |
| control duplex | After thawing, store at 4° C. | |
| Rho siRNA | Store aliquots at −20° C. | Intravitreal |
| | until use. | |
| | After thawing, store at 4° C. | |
| Phenyl-N-tert- | Dry powder stored at RT. | Intraperitoneal |
| butylnitrone (PBN) | Working solutions prepared | |
| | in 0.9% NaCl immediately | |
| | prior to use. | |

Study Arms

TABLE 8

| | | | Treatment | |
|---|---|---|---|---|
| Arm | Housing | Treatment | Details | Assessment |
| 1 | Dark-reared | 0.9% | Bilateral IVT | OKT (P38 and P45); |
| | (P1-30); 12 h | NaCl | (P31) | ERG (P39 and P46); |
| | light/dark | | | Retinal thickness quant |
| | (P31-P46) | | | |
| 2 | Dark-reared | 2 μg | Bilateral IVT | OKT (P38 and P45); |
| | (P1-30); 12 h | Pooled | (P31) | ERG (P39 and P46); |
| | light/dark | mimics | | Retinal thickness quant |
| | (P31-P46) | | | |
| 3 | Dark-reared | 10 μg | Bilateral IVT | OKT (P38 and P45) |
| | (P1-30); 12 h | Pooled | (P31) | ERG (P39 and P46); |
| | light/dark | mimics | | Retinal thickness quant |
| | (P31-P46) | | | |
| 4 | Dark-reared | 10 μg | Bilateral IVT | OKT (P38 and P45); |
| | (P1-30); 12 h | Rho | (P31) | ERG (P39 and P46); |
| | light/dark | siRNA | | Retinal thickness quant |
| | (P31-P46) | | | |
| 5 | Dark-reared | 100 mg/kg | intraperitoneal | OKT (P38 and P45); |
| | (P1-30); 12 h | PBN | administration | ERG (P39 and P46); |
| | light/dark | | (P29-P35) | Retinal thickness quant |
| | (P31-P46) | | | |

Animal Housing

All animals were housed in groups of 3-5 in large cages kept in ventilated shelves under standard
animal care conditions. Rd10/rd10 pregnant dams were housed in darkness upon observation of a mucus plug. Newborn pups were housed with mothers in complete darkness from postnatal day 1 through postnatal day 30. On postnatal day 31, animals were transitioned to maintenance under normal cyclical light conditions consisting of 12 hours of light (<500 lux) followed by 12 hours of darkness.

Formulation Preparation and Storage

Test agents were supplied as aliquots at 10 μg/μl, and were ready to inject. For arm 2, test agent (pooled mimics) was diluted 1:5 in 0.9% NaCl to achieve a final concentration of 2 μg/μl. A total volume of 1 μl was delivered for all intravitreal injections. Aliquots were stored at −20° C. until use. Following the initial thaw, all remaining material was stored at 4° C., and was not refrozen. PBN was prepared immediately prior to use as a 15 mg/ml solution in 0.9% NaCl (Cat #S4041, Teknova). PBN was delivered daily as indicated in Section 3.2 at a dose of 100 mg/kg in a total volume of 75-150 μl, depending on the animal's body weight.

Intraperitoneal (IP) Administration

Sedatives and the positive control test agent (PBN) were delivered by standard techniques for IP injection utilizing a 0.3 cc insulin syringe attached to an 8 mm 31-gauge needle (BD#328438) with a total volume ≦150 μl.

Intravitreal Administration

Animals were anesthetized with ketamine/xylazine using a U-100 syringe utilizing Ketamine (85 mg/kg) and Xylazine (14 mg/kg). Pupils were then dilated with topical administration of Cyclogyl and Ak-Dilate. Following sedation and dilation, a total volume of 1 μl per eye was injected into the vitreous at the pars plana using a Hamilton syringe and a 33 gauge needle.

Optokinetic Tracking (OKT)

All optokinetic tracking experiments are performed using an Optomotry designed for rodent use (Cerebral Mechanics Inc.). In this non-invasive assessment, mice are placed onto a platform surrounded by 4 LCD screens which resides within a light-protected box. Visual stimuli are then presented to the mice via the LCD screens and a masked observer visualizes and scores optokinetic tracking reflexes from a digital camcorder which is mounted on the top of the box. For measurements of spatial frequency threshold, the mice were tested at a range of spatial frequencies from 0.034 to 0.514 cycles/degree. The Optomotry device employs a proprietary algorithm to accept the input from the masked observer and automatically adjust the testing stimuli based upon whether the animal exhibited the correct or incorrect tracking reflex.

Tissue Collection

Following sedation with ketamine/xylazine, animals were euthanized with a lethal dose of pentobarbital. The right eye of all animals was scorched with a flamed needle to demarcate the superior portion of the eye, enucleated, fixed in Z-fix (zinc buffered neutral formalin), and processed for H&E histology. From the left eye of all animals, the retinas were individually isolated, and immediately snap-frozen in liquid N₂, and stored individually in a 2 mL screw cap
polypropylene tube at −70° C. until further processing.

Data and Statistical Analyses

Statistical significance was determined using Prism software (Graphpad Inc.) to perform t-test (OKT and ERG) or one-way Analysis of Variance (ANOVA) calculations (retinal thickness), with a threshold of p<0.05 to determine whether any changes are statistically significant.

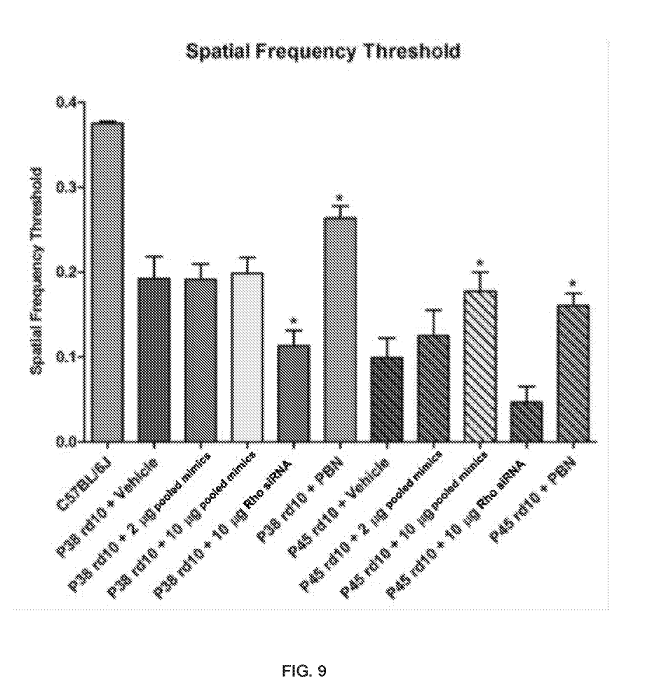

FIG. 9 shows the effect of test agents on visual acuity loss in the mouse model of retinitis pigmentosa. Visual acuity was assessed by the mean SFT at which mice were able to distinguish visual stimuli. Visual acuity was lower in all groups of rd10 mice relative to C57Bl/6J control mice at both P38 and P45. At P38, visual acuity was comparable for the vehicle treated group and both groups receiving pooled mimics. The group receiving 10 μg of Rho siRNA had statistically significant lower visual acuity relative to the vehicle treated group at P38 (p=0.0185; unpaired t test), whereas the PBN treated positive control group displayed statistically significant higher visual acuity measurements relative to vehicle (p=0.0224; unpaired t test). At P45, there is a dose-dependent amelioration in vision loss for the two groups receiving the pooled mimics, with a statistically significant difference between the 10 μg group of the pooled mimics and the vehicle (p=0.0258, unpaired t test). The PBN treated animals also display statistically significant better visual acuity relative to vehicle at P45 (p=0.0416 unpaired t test). The visual acuity for the 10 μg Rho siRNA group remained much lower than the age-matched vehicle group, but the difference was not statistically significant.

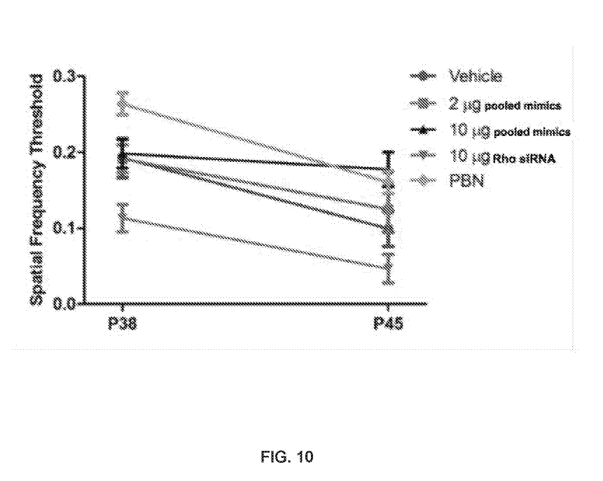

FIG. 10 shows the effect of test agents on visual acuity decline in the mouse model of retinitis pigmentosa. Visual acuity loss occurred across all groups from P38 to P45. Vehicle treated animals exhibit a 48% decrease in visual acuity from P38 to P45. The largest decline in visual acuity across the two time points occurred for the group receiving 10 μg Rho siRNA test agent (59%). The group receiving 10 μg of pooled mimics had the most negligible loss in visual acuity across the two time points (11%). Visual acuity loss was less dramatic for both the 2 μg group of pooled mimics and the PBN group relative to vehicle (35% and 39%, respectively).

At both postnatal time points examined, vision loss assessed by OKT was statistically significantly lower in light exposed rd10/rd10 mice relative to both control C57Bl/6J mice, and rd10/rd10 mice prior to light exposure. The vehicle treated group was lower in its SFT than all groups except the 10 μg Rho siRNA treatment group. Bilateral intravitreal administration of 10 μg of pooled mimics (miR-96, miR-182, and control duplex) demonstrates a positive effect on visual acuity as demonstrated by a statistically significant retention in SFT measurements at P45, and the lowest percentage drop off in SFT from P38 to P45 amongst all groups (FIGS. 9 and 10). The PBN positive control treatment group also retained a statistically significant higher SFT at P45 relative to the vehicle control group, however, the loss in SFT from P38 to P45 was greater in the PBN group than the 10 pig group of pooled mimics. The 2 μg treatment group of pooled mimics had a higher SFT at P45 than the vehicle control group, but the difference was not statistically significant. The group receiving 10 μg Rho siRNA had the largest loss in SFT measurements at both P38 and P45, and also had the largest percentage decline in SFT between the two time points. Based on the data, bilateral intravitreal administration of 10 μg of pooled mimics ameliorates vision loss at the latter time point with a statistically significant difference in visual acuity measured by OKT.

1. A microRNA mimetic compound comprising:
a first strand of about 22 to about 26 ribonucleotides comprising a mature miR-96, miR-182, or miR-183 sequence; and
a second strand of about 20 to about 26 ribonucleotides comprising a sequence that is substantially complementary to the first strand and having at least one modified nucleotide.

2. The microRNA mimetic compound of claim 1, wherein the first strand has one or more 2′ fluoro nucleotides.

3. The microRNA mimetic compound of claim 1, wherein the first strand has no modified nucleotides.

4. The microRNA mimetic compound of any one of claims 1 to 3, wherein the at least one modified nucleotide in the second strand is a 2′-O-methyl modified nucleotide.

5. The microRNA mimetic compound of any one of claims 1 to 4, wherein the second strand is not fully complementary to the first strand.

6. The microRNA mimetic compound of claim 5, wherein the second strand has 1, 2, or 3 mismatches relative to the first strand.

7. The microRNA mimetic compound of any one of claims 1 to 6, wherein the second strand is linked to a cholesterol molecule at its 3′ or 5′ terminus.

8. The microRNA mimetic compound of claim 7, wherein the cholesterol molecule is linked to the second strand through at least a six carbon linker.

9. The microRNA mimetic compound of any one of claims 1 to 8, wherein the first strand has a 5′-terminal monophosphate.

10. The microRNA mimetic compound of any one of claims 1 to 9, wherein the first or the second strand has a 3′ nucleotide overhang relative to the other strand.

11. The microRNA mimetic compound of any one of claims 1 to 10, wherein the nucleotides comprising the 3′ overhang are linked by phosphorothioate linkages.

12. The microRNA mimetic compound of any one of claims 1 to 11, wherein the 3′ nucleotide overhang comprises two ribonucleotides.

13. The microRNA mimetic compound of any one of claims 1 to 12, wherein the first strand comprises a mature miR-96 sequence.

14. The microRNA mimetic compound of claim 13, wherein the first strand comprises a sequence of SEQ ID NO: 10.

15. The microRNA mimetic compound of claim 13 or 14, wherein the second strand comprises a sequence selected from the group consisting of SEQ ID NO: 11, SEQ ID NO: 12, and SEQ ID NO: 13.

16. The microRNA mimetic compound of claim 13, wherein the first strand comprises a sequence selected from the group consisting of SEQ ID NOs: 10 and 26-29 and the second strand comprises a sequence selected from the group consisting of SEQ ID NOs: 11-14 and 30-34.

17. The microRNA mimetic compound of any one of claims 1 to 12, wherein the first strand comprises a mature miR-182 sequence.

18. The microRNA mimetic compound of claim 17, wherein the first strand comprises a sequence of SEQ ID NO: 15.

19. The microRNA mimetic compound of claim 17 or 18, wherein the second strand comprises a sequence selected from the group consisting of SEQ ID NO: 16, SEQ ID NO: 17, and SEQ ID NO: 18.

20. The microRNA mimetic compound of claim 17, wherein the first strand comprises a sequence selected from the group consisting of SEQ ID NOs: 15 and 35-38 and the second strand comprises a sequence selected from the group consisting of SEQ ID NOs: 16-19 and 39-43.

21. The microRNA mimetic compound of any one of claims 1 to 12, wherein the first strand comprises a mature miR-183 sequence.

22. The microRNA mimetic compound of claim 21, wherein the first strand comprises a sequence of SEQ ID NO: 20.

23. The microRNA mimetic compound of claim 21 or 22, wherein the second strand comprises a sequence selected from the group consisting of SEQ ID NO: 21, SEQ ID NO: 22, SEQ ID NO: 23, and SEQ ID NO: 24.

24. The microRNA mimetic compound of claim 21, wherein the first strand comprises a sequence selected from the group consisting of SEQ ID NOs: 20 and 44-47 and the second strand comprises a sequence selected from the group consisting of SEQ ID NOs: 21-25 and 48-52.

25. A pharmaceutical composition comprising a therapeutically effective amount of the microRNA mimetic compound of any one of claims 1 to 24, or a pharmaceutically-acceptable salt thereof, and a pharmaceutically-acceptable carrier or diluent.

26. A pharmaceutical composition comprising a therapeutically effective amount of at least two microRNA mimetic compounds of claim 1, wherein the first strand of the first microRNA mimetic compound comprises a mature miR-96 sequence and the first strand of the second microRNA mimetic compound comprises a mature miR-182 or miR-183 sequence.

27. The pharmaceutical composition of claim 26, wherein the first strand of the second microRNA mimetic compound comprises a mature miR-182 sequence.

28. The pharmaceutical composition of claim 27, further comprising a third microRNA mimetic compound, wherein the first strand of the third microRNA mimetic compound comprises a mature miR-183 sequence.

29. The pharmaceutical composition of claim 28, wherein the first, second, and third microRNA mimetic compounds are present in equimolar concentrations.

30. A method of treating or preventing an ophthalmological condition in a subject in need thereof comprising administering to the subject a therapeutically effective amount of at least one agonist of miR-96, miR-182, and/or miR-183, wherein the agonist is a double-stranded oligonucleotide comprising a first strand comprising a mature miR-96, miR-182, or miR-183 sequence and a second strand comprising a sequence that is substantially complementary to the first strand, wherein at least one of the strands comprises one or more modified nucleotides.

31. The method of claim 30, wherein the therapeutically effective amount is an amount sufficient to maintain or improve visual acuity in the subject.

32. The method of claim 30, wherein the therapeutically effective amount is an amount sufficient to reduce or prevent photoreceptor cell damage and/or death in the subject.

33. The method of any one of claims 30 to 32, wherein the first strand is about 22 to about 26 nucleotides in length and the second strand is about 20 to about 26 nucleotides in length.

34. The method of any one of claims 30 to 33, wherein the first strand has one or more 2′ fluoro nucleotides.

35. The method of any one of claims 30 to 34, wherein the second strand has one or more 2′-O-methyl modified nucleotides.

36. The method of any one of claims 30 to 35, wherein the second strand has 1, 2, or 3 mismatches relative to the first strand.

37. The method of any one of claims 30 to 35, wherein the first or the second strand has a 3′ nucleotide overhang relative to the other strand.

38. The method of claim 37, wherein the nucleotides comprising the 3′ overhang are linked by phosphorothioate linkages.

39. The method of claim 37 or 38, wherein the 3′ nucleotide overhang comprises two ribonucleotides.

40. The method of any one of claims 30 to 39, wherein the second strand is linked to a cholesterol molecule at its 3′ or 5′ terminus.

41. The method of claim 40, wherein the cholesterol molecule is linked to the second strand through at least a six carbon linker.

42. The method of any one of claims 30 to 41, wherein the agonist is a miR-96 agonist and the first strand of the double-stranded oligonucleotide comprises a mature miR-96 sequence.

43. The method of claim 42, wherein the first strand comprises a sequence selected from the group consisting of SEQ ID NOs: 10 and 26-29 and the second strand comprises a sequence selected from the group consisting of SEQ ID NOs: 11-14 and 30-34.

44. The method of any one of claims 30 to 41, wherein the agonist is a miR-182 agonist and the first strand of the double-stranded oligonucleotide comprises a mature miR-182 sequence.

45. The method of claim 44, wherein the first strand comprises a sequence selected from the group consisting of SEQ ID NOs: 15 and 35-38 and the second strand comprises a sequence selected from the group consisting of SEQ ID NOs: 16-19 and 39-43.

46. The method of any one of claims 30 to 41, wherein the agonist is a miR-183 agonist and the first strand of the double-stranded oligonucleotide comprises a mature miR-183 sequence.

47. The method of claim 46, wherein the first strand comprises a sequence selected from the group consisting of SEQ ID NOs: 20 and 44-47 and the second strand comprises a sequence selected from the group consisting of SEQ ID NOs: 21-25 and 48-52.

48. The method of claim 42, further comprising administering to the subject a miR-182 agonist, wherein the miR-182 agonist is a double-stranded oligonucleotide comprising a first strand comprising a mature miR-182 sequence and a second strand comprising a sequence that is substantially complementary to the first strand, wherein at least one of the strands comprises one or more modified nucleotides.

49. The method of claim 42 or 48, further comprising administering to the subject a miR-183 agonist, wherein the miR-183 agonist is a double-stranded oligonucleotide comprising a first strand comprising a mature miR-183 sequence and a second strand comprising a sequence that is substantially complementary to the first strand, wherein at least one of the strands comprises one or more modified nucleotides.

50. The method of claim 49, wherein the miR-96, miR-182, and miR-183 agonists are administered to the subject in separate compositions.

51. The method of claim 49, wherein the miR-96, miR-182, and miR-183 agonists are administered to the subject in the same composition.

52. The method of claim 51, wherein the miR-96, miR-182, and miR-183 agonists are present in the composition at equimolar concentrations.

53. The method of any one of claims 30 to 52, wherein the at least one agonist is administered to the subject ocularly.

54. The method of claim 53, wherein the ocular administration comprises intravitreal, peri-ocular, intracameral, subconjunctival, or transcleral administration.

55. The method of any one of claims 30 to 54, wherein the subject has retinitis pigmentosa.

56. The method of any one of claims 30 to 54, wherein the subject has signs of night blindness.

57. The method of any one of claims 30 to 54, wherein the subject has an ophthalmological condition selected from the group consisting of retinal detachment, retinal degeneration, macular degeneration, and Stargardt disease.

58. The method of any one of claims 30 to 57, wherein the therapeutically effective amount is an amount sufficient to increase expression of one or more phototransduction genes in the photoreceptor cells of the subject.

59. The method of claim 58, wherein the one or more phototransduction genes are selected from Recoverin (Revrn), NRL, Arrestin (Sag), Rhodopsin (Rho), Transducin (Gnat2), and Phosducin (PDC).

60. A method of treating or preventing an ear disorder in a subject in need thereof comprising administering to the subject a therapeutically effective amount of at least one agonist of miR-96, miR-182, and/or miR-183, wherein the agonist is a double-stranded oligonucleotide comprising a first strand comprising a mature miR-96, miR-182, or miR-183 sequence and a second strand comprising a sequence that is substantially complementary to the first strand, wherein at least one of the strands comprises one or more modified nucleotides.

61. The method of claim 60, wherein the ear disorder is selected from the group consisting of hearing loss, tinnitus, Meniere's disease, and ear infections.

62. An expression vector comprising a polynucleotide encoding miR-96, miR-182, or miR-183 for expression in a mammalian cell.

63. The expression vector of claim 62, wherein the vector is a viral expression vector.

64. The expression vector of claim 63, wherein the viral vector is an adeno-associated viral vector.

65. The expression vector of claim 63, wherein the adeno-associated viral vector is a self-complementary adeno-associated viral vector.

66. The expression vector of claim 64 or 65, wherein the adeno-associated viral vector is selected from the group consisting of AAV1, AAV2, AAV3, AAV4, AAV5, AAV6, AAV7, AAV8, and AAV9.

67. The expression vector of claim 62, wherein the expression vector comprises a nucleic acid sequence selected from the group consisting of SEQ ID NOs: 53-55.

68. A method of treating or preventing an ophthalmological condition or an ear condition in a subject in need thereof comprising administering to the subject an effective amount of an expression vector encoding a miR-96, miR-182, and/or miR-183.


SEQUENCE LISTING (55 sequences)

SEQ ID NO: 1 - RNA, 23 nt, Mus sp.
uuuggcacuagcacauuuuugcu

SEQ ID NO: 2 - RNA, 23 nt, Homo sapiens
uuuggcacuagcacauuuuugcu

SEQ ID NO: 3 - RNA, 23 nt, Rattus sp.
uuuggcacuagcacauuuuugcu

SEQ ID NO: 4 - RNA, 25 nt, Mus sp.
uuuggcaaugguagaacucacaccg

SEQ ID NO: 5 - RNA, 24 nt, Homo sapiens
uuuggcaaugguagaacucacacu

SEQ ID NO: 6 - RNA, 25 nt, Rattus sp.
uuuggcaaugguagaacucacaccg

SEQ ID NO: 7 - RNA, 22 nt, Mus sp.
uauggcacugguagaauucacu

SEQ ID NO: 8 - RNA, 22 nt, Homo sapiens
uauggcacugguagaauucacu

SEQ ID NO: 9 - RNA, 22 nt, Rattus sp.
uauggcacugguagaauucacu

SEQ ID NO: 10 - RNA, 25 nt, Artificial Sequence
  miR-96 mimetic compound Antisense_mods_96
  MOD_RES (3)..(3): May be a 2′-fluoronucleotide
  MOD_RES (6)..(6): May be a 2′-fluoronucleotide
  MOD_RES (8)..(9): May be a 2′-fluoronucleotide
  MOD_RES (12)..(12): May be a 2′-fluoronucleotide
  MOD_RES (14)..(14): May be a 2′-fluoronucleotide
  MOD_RES (16)..(20): May be a 2′-fluoronucleotide
  MOD_RES (22)..(23): May be a 2′-fluoronucleotide
  MISC_FEATURE (23)..(25): May be joined by phosphorothioate bonds
uuuggcacuagcacauuuuugcuuu

SEQ ID NO: 11 - RNA, 23 nt, Artificial Sequence
  miR-96 mimetic compound Sense_SS_3MM_96
  MOD_RES (1)..(3): May be a 2′-O-methyl modified nucleotide
  MOD_RES (8)..(9): May be a 2′-O-methyl modified nucleotide
  MOD_RES (13)..(14): May be a 2′-O-methyl modified nucleotide
  MOD_RES (17)..(17): May be a 2′-O-methyl modified nucleotide
  MOD_RES (19)..(19): May be a 2′-O-methyl modified nucleotide
agcaaaauugagcuagugcgaaa

SEQ ID NO: 12 - RNA, 23 nt, Artificial Sequence
  miR-96 mimetic compound Sense_mods__96
  MOD_RES (1)..(3): May be a 2′-O-methyl modified nucleotide
  MOD_RES (9)..(9): May be a 2′-O-methyl modified nucleotide
  MOD_RES (11)..(11): May be a 2′-O-methyl modified nucleotide
  MOD_RES (13)..(14): May be a 2′-O-methyl modified nucleotide
  MOD_RES (17)..(17): May be a 2′-O-methyl modified nucleotide
  MOD_RES (19)..(20): May be a 2′-O-methyl modified nucleotide
agcaaaaaugugcuagugccaaa

SEQ ID NO: 13 - RNA, 23 nt, Artificial Sequence
  miR-96 mimetic compound Sense_SS_3MM_96_Chol_PO
  MOD_RES (1)..(3): May be a 2′-O-methyl modified nucleotide
  MOD_RES (8)..(9): May be a 2′-O-methyl modified nucleotide
  MOD_RES (13)..(14): May be a 2′-O-methyl modified nucleotide
  MOD_RES (17)..(17): May be a 2′-O-methyl modified nucleotide
  MOD_RES (19)..(19): May be a 2′-O-methyl modified nucleotide
  MOD_RES (23)..(23): May be modified with a cholesterol moiety
agcaaaauugagcuagugcgaaa

SEQ ID NO: 14 - RNA, 23 nt, Artificial Sequence
  miR-96 mimetic compound Sense_SS_3MM_96_Chol_PS
  MOD_RES (1)..(3): May be a 2′-O-methyl modified nucleotide
  MOD_RES (8)..(9): May be a 2′-O-methyl modified nucleotide
  MOD_RES (13)..(14): May be a 2′-O-methyl modified nucleotide
  MOD_RES (17)..(17): May be a 2′-O-methyl modified nucleotide
  MOD_RES (19)..(19): May be a 2′-O-methyl modified nucleotide
  MOD_RES (23)..(23): May be modified through a phosphorothioate bond with a cholesterol moiety
agcaaaauugagcuagugcgaaa

SEQ ID NO: 15 - RNA, 26 nt, Artificial Sequence
  miR-182 mimetic compound Antisense_mods_182
  MOD_RES (3)..(3): May be a 2′-fluoronucleotide
  MOD_RES (6)..(6): May be a 2′-fluoronucleotide
  MOD_RES (9)..(9): May be a 2′-fluoronucleotide
  MOD_RES (12)..(12): May be a 2′-fluoronucleotide
  MOD_RES (17)..(19): May be a 2′-fluoronucleotide
  MOD_RES (21)..(21): May be a 2′-fluoronucleotide
  MOD_RES (23)..(24): May be a 2′-fluoronucleotide
  MISC_FEATURE (24)..(26): May be joined by phosphorothioate bonds
uuuggcaaugguagaacucacacuuu

SEQ ID NO: 16 - RNA, 24 nt, Artificial Sequence
  miR-182 mimetic compound Sense_SS_3MM_182
  MOD_RES (1)..(3): May be a 2′-O-methyl modified nucleotide
  MOD_RES (5)..(5): May be a 2′-O-methyl modified nucleotide
  MOD_RES (10)..(11): May be a 2′-O-methyl modified nucleotide
  MOD_RES (14)..(15): May be a 2′-O-methyl modified nucleotide
  MOD_RES (17)..(18): May be a 2′-O-methyl modified nucleotide
  MOD_RES (20)..(20): May be a 2′-O-methyl modified nucleotide
agugugagaucaaccauugcgaaa

SEQ ID NO: 17 - RNA, 24 nt, Artificial Sequence
  miR-182 mimetic compound Sense_mods_182
  MOD_RES (1)..(3): May be a 2′-O-methyl modified nucleotide
  MOD_RES (5)..(5): May be a 2′-O-methyl modified nucleotide
  MOD_RES (9)..(12): May be a 2′-O-methyl modified nucleotide
  MOD_RES (14)..(15): May be a 2′-O-methyl modified nucleotide
  MOD_RES (17)..(18): May be a 2′-O-methyl modified nucleotide
  MOD_RES (20)..(21): May be a 2′-O-methyl modified nucleotide
agugugaguucuaccauugccaaa

SEQ ID NO: 18 - RNA, 24 nt, Artificial Sequence
  miR-182 mimetic compound Sense_SS_3MM_182_Chol_PO
  MOD_RES (1)..(3): May be a 2′-O-methyl modified nucleotide
  MOD_RES (5)..(5): May be a 2′-O-methyl modified nucleotide
  MOD_RES (10)..(11): May be a 2′-O-methyl modified nucleotide
  MOD_RES (14)..(15): May be a 2′-O-methyl modified nucleotide
  MOD_RES (17)..(18): May be a 2′-O-methyl modified nucleotide
  MOD_RES (20)..(20): May be a 2′-O-methyl modified nucleotide
  MOD_RES (24)..(24): May be modified with a cholesterol moiety
agugugagaucaaccauugcgaaa

SEQ ID NO: 19 - RNA, 24 nt, Artificial Sequence
  miR-182 mimetic compound Sense_SS_3MM_182_Chol_PS
  MOD_RES (1)..(3): May be a 2′-O-methyl modified nucleotide
  MOD_RES (5)..(5): May be a 2′-O-methyl modified nucleotide
  MOD_RES (10)..(11): May be a 2′-O-methyl modified nucleotide
  MOD_RES (14)..(15): May be a 2′-O-methyl modified nucleotide
  MOD_RES (17)..(18): May be a 2′-O-methyl modified nucleotide
  MOD_RES (20)..(20): May be a 2′-O-methyl modified nucleotide
  MOD_RES (24)..(24): May be modified through a phosphorothioate bond with a cholesterol moiety
agugugagaucaaccauugcgaaa

SEQ ID NO: 20 - RNA, 24 nt, Artificial Sequence
  miR-183 mimetic compound Antisense_mods_183
  MOD_RES (3)..(3): May be a 2′-fluoronucleotide
  MOD_RES (6)..(6): May be a 2′-fluoronucleotide
  MOD_RES (8)..(9): May be a 2′-fluoronucleotide
  MOD_RES (12)..(12): May be a 2′-fluoronucleotide
  MOD_RES (17)..(19): May be a 2′-fluoronucleotide
  MOD_RES (21)..(22): May be a 2′-fluoronucleotide
  MISC_FEATURE (22)..(24): May be joined by phosphorothioate bonds
uauggcacugguagaauucacuuu

SEQ ID NO: 21 - RNA, 22 nt, Artificial Sequence
  miR-183 mimetic compound Sense_SS_3MM_183
  MOD_RES (1)..(3): May be a 2′-O-methyl modified nucleotide
  MOD_RES (8)..(9): May be a 2′-O-methyl modified nucleotide
  MOD_RES (12)..(13): May be a 2′-O-methyl modified nucleotide
  MOD_RES (16)..(16): May be a 2′-O-methyl modified nucleotide
  MOD_RES (18)..(18): May be a 2′-O-methyl modified nucleotide
  MOD_RES (21)..(21): May be a 2′-O-methyl modified nucleotide
agugaaaucaaccagugcgaua

SEQ ID NO: 22 - RNA, 22 nt, Artificial Sequence
  miR-183 mimetic compound Sense_mods_183
  MOD_RES (1)..(3): May be a 2′-O-methyl modified nucleotide
  MOD_RES (7)..(10): May be a 2′-O-methyl modified nucleotide
  MOD_RES (12)..(13): May be a 2′-O-methyl modified nucleotide
  MOD_RES (16)..(16): May be a 2′-O-methyl modified nucleotide
  MOD_RES (18)..(19): May be a 2′-O-methyl modified nucleotide
  MOD_RES (21)..(21): May be a 2′-O-methyl modified nucleotide
agugaauucuaccagugccaua

SEQ ID NO: 23 - RNA, 22 nt, Artificial Sequence
  miR-183 mimetic compound Sense_SS_3MM_183_Chol_PO
  MOD_RES (1)..(3): May be a 2′-O-methyl modified nucleotide
  MOD_RES (8)..(9): May be a 2′-O-methyl modified nucleotide
  MOD_RES (12)..(13): May be a 2′-O-methyl modified nucleotide
  MOD_RES (16)..(16): May be a 2′-O-methyl modified nucleotide
  MOD_RES (18)..(18): May be a 2′-O-methyl modified nucleotide
  MOD_RES (21)..(21): May be a 2′-O-methyl modified nucleotide
  MOD_RES (22)..(22): May be modified with a cholesterol moiety
agugaaaucaaccagugcgaua

SEQ ID NO: 24 - RNA, 22 nt, Artificial Sequence
  miR-183 mimetic compound Sense_mods_183_Chol_PO
  MOD_RES (1)..(3): May be a 2′-O-methyl modified nucleotide
  MOD_RES (7)..(10): May be a 2′-O-methyl modified nucleotide
  MOD_RES (12)..(13): May be a 2′-O-methyl modified nucleotide
  MOD_RES (16)..(16): May be a 2′-O-methyl modified nucleotide
  MOD_RES (18)..(19): May be a 2′-O-methyl modified nucleotide
  MOD_RES (21)..(21): May be a 2′-O-methyl modified nucleotide
  MOD_RES (22)..(22): May be modified with a cholesterol moiety
agugaauucuaccagugccaua

SEQ ID NO: 25 - RNA, 22 nt, Artificial Sequence
  miR-183 mimetic compound Sense_SS_3MM_183_Chol_PS
  MOD_RES (1)..(3): May be a 2′-O-methyl modified nucleotide
  MOD_RES (8)..(9): May be a 2′-O-methyl modified nucleotide
  MOD_RES (12)..(13): May be a 2′-O-methyl modified nucleotide
  MOD_RES (16)..(16): May be a 2′-O-methyl modified nucleotide
  MOD_RES (18)..(18): May be a 2′-O-methyl modified nucleotide
  MOD_RES (21)..(21): May be a 2′-O-methyl modified nucleotide
  MOD_RES (22)..(22): May be modified through a phosphorothioate bond with a cholesterol moiety
agugaaaucaaccagugcgaua

SEQ ID NO: 26 - RNA, 23 nt, Artificial Sequence
  miR-96 mimetic compound Antisense_blunt_96
  MOD_RES (3)..(3): May be a 2′-fluoronucleotide
  MOD_RES (6)..(6): May be a 2′-fluoronucleotide
  MOD_RES (8)..(9): May be a 2′-fluoronucleotide
  MOD_RES (12)..(12): May be a 2′-fluoronucleotide
  MOD_RES (14)..(14): May be a 2′-fluoronucleotide
  MOD_RES (16)..(20): May be a 2′-fluoronucleotide
  MOD_RES (22)..(23): May be a 2′-fluoronucleotide
uuuggcacuagcacauuuuugcu

SEQ ID NO: 27 - RNA, 25 nt, Artificial Sequence
  miR-96 mimetic compound Antisense_mods_5P_96
  MOD_RES (1)..(1): May be modified with a phosphate moiety
  MOD_RES (3)..(3): May be a 2′-fluoronucleotide
  MOD_RES (8)..(9): May be a 2′-fluoronucleotide
  MOD_RES (12)..(12): May be a 2′-fluoronucleotide
  MOD_RES (14)..(14): May be a 2′-fluoronucleotide
  MOD_RES (16)..(20): May be a 2′-fluoronucleotide
  MOD_RES (22)..(23): May be a 2′-fluoronucleotide
  MISC_FEATURE (24)..(25): May be joined by phosphorothioate bonds
uuuggcacuagcacauuuuugcuuu

SEQ ID NO: 28 - RNA, 25 nt, Artificial Sequence
  miR-96 mimetic compound Antisense_unmod_96
uuuggcacuagcacauuuuugcuuu

SEQ ID NO: 29 - RNA, 25 nt, Artificial Sequence
  miR-96 mimetic compound Antisense_2′OMe_96
  MOD_RES (3)..(3): May be a 2′-O-methyl modified nucleotide
  MOD_RES (6)..(6): May be a 2′-O-methyl modified nucleotide
  MOD_RES (8)..(9): May be a 2′-O-methyl modified nucleotide
  MOD_RES (12)..(12): May be a 2′-O-methyl modified nucleotide
  MOD_RES (14)..(14): May be a 2′-O-methyl modified nucleotide
  MOD_RES (16)..(20): May be a 2′-O-methyl modified nucleotide
  MOD_RES (22)..(23): May be a 2′-O-methyl modified nucleotide
  MISC_FEATURE (24)..(25): May be joined by phosphorothioate bonds
uuuggcacuagcacauuuuugcuuu

SEQ ID NO: 30 - RNA, 25 nt, Artificial Sequence
  miR-96 mimetic compound Sense_OH_96
  MOD_RES (1)..(3): May be a 2′-O-methyl modified nucleotide
  MOD_RES (8)..(9): May be a 2′-O-methyl modified nucleotide
  MOD_RES (13)..(13): May be a 2′-O-methyl modified nucleotide
  MOD_RES (14)..(14): May be a 2′-O-methyl modified nucleotide
  MOD_RES (17)..(17): May be a 2′-O-methyl modified nucleotide
  MOD_RES (19)..(19): May be a 2′-O-methyl modified nucleotide
  MISC_FEATURE (23)..(25): May be joined by phosphorothioate bonds
agcaaaauugagcuagugcgaaauu

SEQ ID NO: 31 - RNA, 23 nt, Artificial Sequence
  miR-96 mimetic compound Sense_SS_3MM_5′Chol_96
  MOD_RES (1)..(1): May be modified with a cholesterol attached to the oligo via a 6-carbon linker
  MOD_RES (1)..(3): May be a 2′-O-methyl modified nucleotide
  MOD_RES (8)..(9): May be a 2′-O-methyl modified nucleotide
  MOD_RES (13)..(14): May be a 2′-O-methyl modified nucleotide
  MOD_RES (17)..(17): May be a 2′-O-methyl modified nucleotide
  MOD_RES (19)..(19): May be a 2′-O-methyl modified nucleotide
agcaaaauugagcuagugcgaaa

SEQ ID NO: 32 - RNA, 25 nt, Artificial Sequence
  miR-96 mimetic compound Sense_OT_96
  MOD_RES (1)..(2): May be a 2′-O-methyl modified nucleotide
agcaaaaaugugcuagugccaaauu

SEQ ID NO: 33 - RNA, 23 nt, Artificial Sequence
  miR-96 mimetic compound Sense_SS_3MM_96_Chol
  MOD_RES (1)..(3): May be a 2′-O-methyl modified nucleotide
  MOD_RES (8)..(9): May be a 2′-O-methyl modified nucleotide
  MOD_RES (13)..(14): May be a 2′-O-methyl modified nucleotide
  MOD_RES (17)..(17): May be a 2′-O-methyl modified nucleotide
  MOD_RES (19)..(19): May be a 2′-O-methyl modified nucleotide
  MOD_RES (23)..(23): May be modified through a phosphorothioate bond with a cholesterol moiety
agcaaaauugagcuagugcgaaa

SEQ ID NO: 34 - RNA, 23 nt, Artificial Sequence
  miR-96 mimetic compound Sense_SS_3MM_96_Chol_PO
  MOD_RES (1)..(3): May be a 2′-O-methyl modified nucleotide
  MOD_RES (8)..(9): May be a 2′-O-methyl modified nucleotide
  MOD_RES (13)..(14): May be a 2′-O-methyl modified nucleotide
  MOD_RES (17)..(17): May be a 2′-O-methyl modified nucleotide
  MOD_RES (19)..(19): May be a 2′-O-methyl modified nucleotide
  MOD_RES (23)..(23): May be modified with a cholesterol moiety
agcaaaauugagcuagugcgaaa

SEQ ID NO: 35 - RNA, 24 nt, Artificial Sequence
  miR-182 mimetic compound Antisense_blunt_182
  MOD_RES (3)..(3): May be a 2′-fluoronucleotide
  MOD_RES (6)..(6): May be a 2′-fluoronucleotide
  MOD_RES (9)..(9): May be a 2′-fluoronucleotide
  MOD_RES (12)..(12): May be a 2′-fluoronucleotide
  MOD_RES (17)..(19): May be a 2′-fluoronucleotide
  MOD_RES (21)..(21): May be a 2′-fluoronucleotide
  MOD_RES (23)..(24): May be a 2′-fluoronucleotide
uuuggcaaugguagaacucacacu

SEQ ID NO: 36 - RNA, 26 nt, Artificial Sequence
  miR-182 mimetic compound Antisense_mods_5P_182
  MOD_RES (1)..(1): May be modified with a phosphate moiety
  MOD_RES (3)..(3): May be a 2′-fluoronucleotide
  MOD_RES (6)..(6): May be a 2′-fluoronucleotide
  MOD_RES (9)..(9): May be a 2′-fluoronucleotide
  MOD_RES (12)..(12): May be a 2′-fluoronucleotide
  MOD_RES (17)..(19): May be a 2′-fluoronucleotide
  MOD_RES (21)..(21): May be a 2′-fluoronucleotide
  MOD_RES (23)..(24): May be a 2′-fluoronucleotide
  MISC_FEATURE (25)..(26): May be joined by phosphorothioate bonds
uuuggcaaugguagaacucacacuuu

SEQ ID NO: 37 - RNA, 26 nt, Artificial Sequence
  miR-182 mimetic compound Antisense_unmod_182
uuuggcaaugguagaacucacacuuu

SEQ ID NO: 38 - RNA, 26 nt, Artificial Sequence
  miR-182 mimetic compound Antisense_2′Ome_182
  MOD_RES (3)..(3): May be a 2′-O-methyl modified nucleotide
  MOD_RES (6)..(6): May be a 2′-O-methyl modified nucleotide
  MOD_RES (9)..(9): May be a 2′-O-methyl modified nucleotide
  MOD_RES (12)..(12): May be a 2′-O-methyl modified nucleotide
  MOD_RES (17)..(19): May be a 2′-O-methyl modified nucleotide
  MOD_RES (21)..(21): May be a 2′-O-methyl modified nucleotide
  MOD_RES (23)..(24): May be a 2′-O-methyl modified nucleotide
  MISC_FEATURE (25)..(26): May be joined by phosphorothioate bonds
uuuggcaaugguagaacucacacuuu

SEQ ID NO: 39 - RNA, 26 nt, Artificial Sequence
  miR-182 mimetic compound Sense_OH_182
  MOD_RES (1)..(3): May be a 2′-O-methyl modified nucleotide
  MOD_RES (5)..(5): May be a 2′-O-methyl modified nucleotide
  MOD_RES (10)..(11): May be a 2′-O-methyl modified nucleotide
  MOD_RES (14)..(15): May be a 2′-O-methyl modified nucleotide
  MOD_RES (17)..(18): May be a 2′-O-methyl modified nucleotide
  MOD_RES (20)..(20): May be a 2′-O-methyl modified nucleotide
  MISC_FEATURE (24)..(26): May be joined by phosphorothioate bonds
agugugagaucaaccauugcgaaauu

SEQ ID NO: 40 - RNA, 24 nt, Artificial Sequence
  miR-182 mimetic compound Sense_SS_3MM_5′chol_182
  MOD_RES (1)..(1): May be modified with a cholesterol attached to the oligo via a 6-carbon linker
  MOD_RES (1)..(3): May be a 2′-O-methyl modified nucleotide
  MOD_RES (5)..(5): May be a 2′-O-methyl modified nucleotide
  MOD_RES (10)..(11): May be a 2′-O-methyl modified nucleotide
  MOD_RES (14)..(15): May be a 2′-O-methyl modified nucleotide
  MOD_RES (17)..(18): May be a 2′-O-methyl modified nucleotide
  MOD_RES (20)..(20): May be a 2′-O-methyl modified nucleotide
agugugagaucaaccauugcgaaa

SEQ ID NO: 41 - RNA, 26 nt, Artificial Sequence
  miR-182 mimetic compound Sense_OT_182
  MOD_RES (1)..(2): May be a 2′-O-methyl modified nucleotide
agugugaguucuaccauugccaaauu

SEQ ID NO: 42 - RNA, 24 nt, Artificial Sequence
  miR-182 mimetic compound Sense_SS_3MM_182_Chol
  MOD_RES (1)..(3): May be a 2′-O-methyl modified nucleotide
  MOD_RES (5)..(5): May be a 2′-O-methyl modified nucleotide
  MOD_RES (10)..(11): May be a 2′-O-methyl modified nucleotide
  MOD_RES (14)..(15): May be a 2′-O-methyl modified nucleotide
  MOD_RES (17)..(18): May be a 2′-O-methyl modified nucleotide
  MOD_RES (20)..(20): May be a 2′-O-methyl modified nucleotide
  MOD_RES (24)..(24): May be modified through a phosphorothioate bond with a cholesterol moiety
agugugagaucaaccauugcgaaa

SEQ ID NO: 43 - RNA, 24 nt, Artificial Sequence
  miR-182 mimetic compound Sense_SS_3MM_182_Chol_PO
  MOD_RES (1)..(3): May be a 2′-O-methyl modified nucleotide
  MOD_RES (5)..(5): May be a 2′-O-methyl modified nucleotide
  MOD_RES (10)..(11): May be a 2′-O-methyl modified nucleotide
  MOD_RES (14)..(15): May be a 2′-O-methyl modified nucleotide
  MOD_RES (17)..(18): May be a 2′-O-methyl modified nucleotide
  MOD_RES (20)..(20): May be a 2′-O-methyl modified nucleotide
  MOD_RES (24)..(24): May be modified with a cholesterol moiety
agugugagaucaaccauugcgaaa

SEQ ID NO: 44 - RNA, 22 nt, Artificial Sequence
  miR-183 mimetic compound Antisense_blunt_183
  MOD_RES (3)..(3): May be a 2′-fluoronucleotide
  MOD_RES (6)..(6): May be a 2′-fluoronucleotide
  MOD_RES (8)..(9): May be a 2′-fluoronucleotide
  MOD_RES (12)..(12): May be a 2′-fluoronucleotide
  MOD_RES (17)..(19): May be a 2′-fluoronucleotide
  MOD_RES (21)..(22): May be a 2′-fluoronucleotide
uauggcacugguagaauucacu

SEQ ID NO: 45 - RNA, 24 nt, Artificial Sequence
  miR-183 mimetic compound Antisense2_mods_5P_183
  MOD_RES (1)..(1): May be modified with a phosphate moiety
  MOD_RES (3)..(3): May be a 2′-fluoronucleotide
  MOD_RES (6)..(6): May be a 2′-fluoronucleotide
  MOD_RES (8)..(9): May be a 2′-fluoronucleotide
  MOD_RES (12)..(12): May be a 2′-fluoronucleotide
  MOD_RES (17)..(19): May be a 2′-fluoronucleotide
  MOD_RES (21)..(22): May be a 2′-fluoronucleotide
  MISC_FEATURE (23)..(24): May be joined by phosphorothioate bonds
uauggcacugguagaauucacuuu

SEQ ID NO: 46 - RNA, 24 nt, Artificial Sequence
  miR-183 mimetic compound Antisense2_unmod_183
uauggcacugguagaauucacuuu

SEQ ID NO: 47 - RNA, 24 nt, Artificial Sequence
  miR-183 mimetic compound Antisense_2′Ome_183
  MOD_RES (3)..(3): May be a 2′-O-methyl modified nucleotide
  MOD_RES (6)..(6): May be a 2′-O-methyl modified nucleotide
  MOD_RES (8)..(9): May be a 2′-O-methyl modified nucleotide
  MOD_RES (12)..(12): May be a 2′-O-methyl modified nucleotide
  MOD_RES (17)..(19): May be a 2′-O-methyl modified nucleotide
  MOD_RES (21)..(22): May be a 2′-O-methyl modified nucleotide
  MISC_FEATURE (23)..(24): May be joined by phosphorothioate bonds
uauggcacugguagaauucacuuu

SEQ ID NO: 48 - RNA, 24 nt, Artificial Sequence
  miR-183 mimetic compound Sense_OH_183
  MOD_RES (1)..(3): May be a 2′-O-methyl modified nucleotide
  MOD_RES (7)..(10): May be a 2′-O-methyl modified nucleotide
  MOD_RES (12)..(13): May be a 2′-O-methyl modified nucleotide
  MOD_RES (16)..(16): May be a 2′-O-methyl modified nucleotide
  MOD_RES (18)..(19): May be a 2′-O-methyl modified nucleotide
  MOD_RES (21)..(21): May be a 2′-O-methyl modified nucleotide
  MISC_FEATURE (22)..(24): May be joined by phosphorothioate bonds
agugaauucuaccagugccauauu

SEQ ID NO: 49 - RNA, 22 nt, Artificial Sequence
  miR-183 mimetic compound Sense2_SS_3MM_5′chol_183
  MOD_RES (1)..(1): May be modified with a cholesterol attached to the oligo via a 6-carbon linker
  MOD_RES (1)..(3): May be a 2′-O-methyl modified nucleotide
  MOD_RES (8)..(9): May be a 2′-O-methyl modified nucleotide
  MOD_RES (12)..(13): May be a 2′-O-methyl modified nucleotide
  MOD_RES (16)..(16): May be a 2′-O-methyl modified nucleotide
  MOD_RES (18)..(18): May be a 2′-O-methyl modified nucleotide
  MOD_RES (21)..(21): May be a 2′-O-methyl modified nucleotide
agugaaaucaaccagugcgaua

SEQ ID NO: 50 - RNA, 24 nt, Artificial Sequence
  miR-183 mimetic compound Sense_OT_183
  MOD_RES (1)..(2): May be a 2′-O-methyl modified nucleotide
agugaauucuaccagugccauauu

SEQ ID NO: 51 - RNA, 22 nt, Artificial Sequence
  miR-183 mimetic compound Sense2_SS_3MM_183_Chol_PO
  MOD_RES (1)..(3): May be a 2′-O-methyl modified nucleotide
  MOD_RES (8)..(9): May be a 2′-O-methyl modified nucleotide
  MOD_RES (12)..(13): May be a 2′-O-methyl modified nucleotide
  MOD_RES (16)..(16): May be a 2′-O-methyl modified nucleotide
  MOD_RES (18)..(18): May be a 2′-O-methyl modified nucleotide
  MOD_RES (21)..(21): May be a 2′-O-methyl modified nucleotide
  MOD_RES (22)..(22): May be modified with a cholesterol moiety
agugaaaucaaccagugcgaua

SEQ ID NO: 52 - RNA, 22 nt, Artificial Sequence
  miR-183 mimetic compound Sense2_SS_3MM_183_Chol
  MOD_RES (1)..(3): May be a 2′-O-methyl modified nucleotide
  MOD_RES (8)..(9): May be a 2′-O-methyl modified nucleotide
  MOD_RES (12)..(13): May be a 2′-O-methyl modified nucleotide
  MOD_RES (16)..(16): May be a 2′-O-methyl modified nucleotide
  MOD_RES (18)..(18): May be a 2′-O-methyl modified nucleotide
  MOD_RES (21)..(21): May be a 2′-O-methyl modified nucleotide
  MOD_RES (22)..(22): May be modified through a phosphorothioate bond with a cholesterol moiety
agugaaaucaaccagugcgaua

SEQ ID NO: 53 - DNA, 110 bp, Homo sapiens
gagctgcttgcctccccccgtttttggcaatggtagaactcacactggtgaggtaacaggatccggtggttctagacttgccaactatggggcgaggactcagccggcac

SEQ ID NO: 54 - DNA, 110 bp, Homo sapiens
ccgcagagtgtgactcctgttctgtgtatggcactggtagaattcactgtgaacagtctcagtcagtgaattaccgaagggccataaacagagcagagacagatccacga

SEQ ID NO: 55 - DNA, 78 bp, Homo sapiens
tggccgattttggcactagcacatttttgcttgtgtctctccgctctgagcaatcatgtgcagtgccaatatgggaaa